SEQUENCE LISTING (17 sequences)

SEQ ID NO: 1 - DNA, 7347 bp, Homo Sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcagaaacctttgaaagcactccgattcgaggagccctgcagattgaaagcagtgaggaaaccgaccagggcaaatatgagtgtgtggccaccaacagcgccggcgtgcgctactcctcacctgccaacctctacgtgcgagagcttcgagaagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccaaaccgtcagccccccctcaagacgttaaatgtgtcagcgtgcgctccacggccattttggtaagttggcgcccgccgccgccggaaacgcacaacggggccctggtgggctacagcgtccgctaccgaccgctgggctcagaggacccggaacccaaggaggtgaacggcatccccccgaccaccactcagatcctgctggaggccttggagaagtggacccagtaccgcatcacgactgtcgctcacacagaggtgggaccagggcccgagagctcgcccgtggtcgtccgcaccgacgaggatgtgcccagcgcgccgccgcggaaggtggaggcggaggcgctcaacgccacggccatccgcgtgctgtggcgctcgcccgcgcccggccggcagcacggccagatccgcggctaccaggtccactacgtgcgcatggagggcgccgaggcccgcgggccgccgcgcatcaaggacgtcatgctggccgatgcccagtgggagacggatgacacggccgaatatgagatggtcatcacaaacttgcagcctgagaccgcgtactccatcacggtagccgcctacaccatgaagggcgatggcgctcgcagcaaacccaaggtggttgtgaccaagggagcagtgctgggccgcccaaccctgtcggtgcagcagacccccgagggcagcctgctggcacgctgggagcccccggctggcaccgcggaggaccaggtgctgggctaccgcctgcagtttggccgtgaggactcgacgcccctggccaccctggagttcccgccctccgaggaccgctacacggcatcaggcgtgcacaagggggccacgtatgtgttccggcttgcggcccggagccgcggcggcctgggcgaggaggcagccgaggtcctgagcatcccggaggacacgccccgtggccacccgcagattctggaggcggccggcaacgcctcggccgggaccgtccttctccgctggctgccacccgtgcccgccgagcgcaacggggccatcgtcaaatacacggtggccgtgcgggaggccggtgccctgggccctgcccgagagactgagctgccggcagcggctgagccgggcgcggagaacgcgctcacgctgcagggcctgaagcccgacacggcctatgacctccaagtgcgagcccacacgcgccggggccctggccccttcagcccccccgtccgctaccggacgttcctgcgggaccaagtctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccagattcaggcctcaggagccccctcagggagccggggtttcactttgaaagcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 2 - protein, 1948 aa, Homo Sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 3 - DNA, 21 bp, Homo Sapiens
cacggcatcaggcgtgcacaa

SEQ ID NO: 4 - DNA, 21 bp, Homo Sapiens
cgcgtctactacaccatggaa

SEQ ID NO: 5 - DNA, 21 bp, Homo Sapiens
caggacattctctctgcacaa

SEQ ID NO: 6 - DNA, 21 bp, Homo Sapiens
aagaacaaacccgacagtaaa

SEQ ID NO: 7 - DNA, 21 bp, Homo Sapiens
cacaggctgctttatcgtcat

SEQ ID NO: 8 - DNA, 21 bp, Artificial Sequence
  SiRNA sense strand targeting the oligonucleotide described by Seq Id No. 3
cggcaucaggcgugcacaatt

SEQ ID NO: 9 - DNA, 21 bp, Artificial Sequence
  SiRNA antisense strand targeting the oligonucleotide described by Seq Id No. 3
uugugcacgccugaugccgtg

SEQ ID NO: 10 - DNA, 21 bp, Artificial Sequence
  SiRNA sense strand targeting the oligonucleotide described by Seq Id No. 4
cgucuacuacaccauggaatt

SEQ ID NO: 11 - DNA, 21 bp, Artificial Sequence
  SiRNA antisense strand targeting the oligonucleotide described by Seq Id No. 4
uuccaugguguaguagacgtg

SEQ ID NO: 12 - DNA, 21 bp, Artificial Sequence
  SiRNA sense strand targeting the oligonucleotide described by Seq. Id No. 5
ggacauucucucugcacaatt

SEQ ID NO: 13 - DNA, 21 bp, Artificial Sequence
  SiRNA antisense strand targeting the oligonucleotide described by Seq. Id No. 5
uugugcagagagaaugucctg

SEQ ID NO: 14 - DNA, 21 bp, Artificial Sequence
  SiRNA sense strand targeting the oligonucleotide described by Seq. Id No. 6
gaacaaacccgacaguaaatt

SEQ ID NO: 15 - DNA, 21 bp, Artificial Sequence
  SiRNA antisense strand targeting the oligonucleotide described by Seq. Id No. 6
uuuacugucggguuuguuctg

SEQ ID NO: 16 - DNA, 18 bp, Artificial Seqeunce
  SiRNA sense strand targeting the oligonucleotide described by Seq. Id No. 7
caggcuuuaucgucautt

SEQ ID NO: 17 - DNA, 21 bp, Artificial Sequence
  SiRNA antisense strand targeting the oligonucleotide described in Seq. Id No. 7
augacgauaaagcagccugtg

(19) United States
(12) Patent Application Publication
(10) Pub. No.: US 2012/0045459 A1
(43) Pub. Date: Feb. 23, 2012

(54) Methods for Treating Autophagy-Related Disorders

(75) Inventors: Jeffrey Paul MacKeigan, East Grand Rapids, MI (US); Katie Martin, Minneapolis, MN (US); Huaqiang Eric Xu, Grand Rapids, MI (US); Yong Xu, Guangzhou (CN)

(73) Assignee: Van Andel Research Institute, Grand Rapids, MI (US)

(21) Appl. No.: 13/318,165

(22) PCT Filed: May 5, 2010
(86) PCT No.: PCT/US10/33740

§ 371 Date: Oct. 31, 2011

Related U.S. Application Data
(62) Division of application No. 61/175,657, filed on May 5, 2009.

(51) Int. Cl. A61K 39/395; A61K 31/18; A61K 31/415; A61K 31/216; C12N 5/07; C12N 9/99; G01N 33/53; G01N 33/68; G01N 33/50; A61K 31/711; A61P 25/28; C12Q 1/68; A61P 9/00; A61P 3/00; A61P 35/00; A61P 37/00; A61P 21/00; A61K 31/7105

(52) U.S. Cl. 424/172.1; 514/44 A; 514/601; 514/403; 514/546; 435/375; 435/184; 435/7.21; 436/86; 436/94; 435/6.12; 435/6.13

(57) ABSTRACT

Methods for treating autophagy-related disorders with agents which modulate expression of the gene encoding tyrosine phosphatase receptor type sigma (PTPRS) or which modulate the biological activity of the PTPRS gene product (PTPsigma). Methods for modulating autophagy in a cell with agents which modulate expression of PTPRS or which modulate the biological activity of PTPsigma; and related diagnostic methods, screening methods, and agents.

### CROSS REFERENCE TO RELATED APPLICATIONS

This application claims priority to U.S. Provisional Application No. 61/175,657, filed on May 5, 2009, the contents of which are incorporated by reference in its entirety.

### INCORPORATION BY REFERENCE OF MATERIAL SUBMITTED ELECTRONICALLY

Incorporated by reference in its entirety is a computer-readable Sequence Listing submitted concurrently herewith and identified as a 30,312 byte ASCII (Text) file named “VAN067FP410WOSequenceListing_ST25.txt,” created on May 5, 2010, and containing material identified as SEQ ID NOS: 1-17.

### FIELD OF THE INVENTION

The invention is in the field of biochemistry and medicine and relates to methods and agents for modulating autophagy disorders.

### BACKGROUND OF THE INVENTION

In addition to the well-characterized role of PI(3)P in endocytosis, recent evidence has uncovered a critical requirement for this lipid in autophagy. Autophagy occurs constitutively in nearly all cells to maintain cellular homeostasis, but it is dramatically activated in response to cellular stress as a survival or adaptation mechanism. Vps34, in complex with Vps15, Beclin, UVRAG, and Bif1, generates PI(3)P on the phagophore, which in turn recruits and tethers effector proteins such as Atg18. The phagophore expands as it sequesters cargo, fuses into a double-membrane autophagosome, and delivers its contents to the lysosome for degradation. Basic biochemical components (i.e., amino acids and fatty acids) are exported from the lysosome and reused by the cell, representing an energetically favorable alternative to de novo synthesis. The critical requirement for PI(3)P in this process is evidenced by the fact that autophagy is ablated in mutant Vps34 yeast strains and genetic Vps34 knockouts in Drosophila. The antagonistic phosphatases which regulate PI(3)P during autophagy are unclear. Several myotubularin-related phosphatases (MTMs) harbor PI(3)P and PI(3,5)P₂ phosphatase activity in vitro and serve important functions in endocytosis, but their role in autophagy (if any) is unclear.

The critical function of phosphatases in lipid signaling is exemplified by the pivotal role of the lipid phosphatase PTEN (phosphatase and tensin homolog) in controlling cell survival, proliferation, and growth. Despite its homology to protein tyrosine phosphatases (PTPs), PTEN dephosphorylates PI(3,4,5)P₃ in vivo and potently antagonizes the action of class I PI3Ks. The tumor suppressive function of PTEN underscores the importance of identifying and characterizing phosphatases that similarly regulate PI(3)P and autophagy.

### SUMMARY OF THE INVENTION

Macroautophagy is a dynamic process whereby portions of the cytosol are encapsulated in double-membrane vesicles and delivered to the lysosome for degradation. Phosphatidylinositol-3-phosphate (PI(3)P) is generated on the earliest autophagic membrane (phagophore) and recruits effector proteins critical for this process. The production of PI(3)P by the class III PI3-kinase Vps34 has been well established; however, phosphatases which dephosphorylate this lipid during autophagy are unknown. To identify such enzymes, the inventors screened human phosphatase genes by RNA interference (RNAi) and found that loss of PTPsigma, a dual-domain protein tyrosine phosphatase (PTP), increases cellular PI(3)P and hyperactivates autophagy. This autophagic phenotype was confirmed in Ptprs−/− MEFs when compared with wild-type counterparts. Further, the inventors discovered that this classically defined PTP harbors lipid phosphatase activity and its active site binds PI(3)P. The inventors findings suggest a novel role for PTPsigma and provide insight into the regulation of autophagy. Mechanistic knowledge of this process is critical for understanding and targeting therapies for several human diseases, including Alzheimer's disease and cancer, in which abnormal autophagy may be pathological. Finally, the inventors' results establish the possibility that other dual-domain PTPs may similarly function as binary function phosphatases, phosphatases that use both phosphoproteins and phospholipids as substrates.

The present invention includes a method of treating an autophagy-related disorder in a subject, comprising administering to the subject an effective amount of an agent which modulates expression of the gene encoding protein tyrosine phosphatase receptor type sigma (PTPRS) or the PTPRS gene product (PTPsigma), or which modulates the biological activity of the PTPsigma. In further embodiments of the present invention: the agent may be an antagonist or an agonist; the biological activity which is modulated may be the phosphatase activity of PTPsigma; or the agent may disrupt the interaction between PTPsigma and phosphatidylinositol 3-phosphate [PI(3)P] or phosphotyrosine (p-Tyr) protein.

The present inventive method are directed against autophagy-related disorders that may include a neurodegenerative disorder, an auto-immune disorder, a cardiovascular disorder, a metabolic disorder, hamartoma syndrome, a genetic muscle disorder, a myopathy, and a cancer.

Further, agents that may be used in implementing the present invention may include an inhibitory nucleic acid, a small organic molecule, an anti-PTPsigma antibody or antigen-binding fragment thereof, or and derivatives thereof. In one embodiment, the agent may be an inhibitory nucleic acid selected from the group consisting of an siRNA targeting any one of the nucleic acids of SEQ ID NOs: 3-7.

In another embodiment the agent for treating an autophagy-related disorder may be a small organic molecule. One example of such an agent is a sulfonamide of the formula:

R₁—NH—SO₂—R₂—O—(CH₂)ₙ—CO—NR₃R₄ (I)

where n is 1 thru 3;

where R₁ is:
C₁-C₄ alkyl;
C₃-C₇ cycloalkyl;
phenyl-(CH₂)ₘ— where m is 0 thru 2 and phenyl is optionally substituted with one or two CH₃—, C₂H₅—, F— and Cl—;
phenyl-CH(CH₃)— where phenyl is optionally substituted with CH₃—, C₂H₅—, F— and Cl—;

where R₂ is phenyl optionally substituted with one F—, Cl—, CH₃—, C₂H₅—, and (CH₃)₂CH—;

where R₃ is H—:

where R₄ is:
C₁-C₃ alkyl;
C₃-C₇ cycloalkyl;
—CH₂—CH═CH₂;
—(CH₂)_{z}—O—R₅ where z is 1 thru 5 and R₅ is C₁-C₃ alkyl;
—(CH₂)_{w}—R₆ where w is 1 thru 3 and R₆ is tetrahydrofuran or C₃-C₇ cycloalkyl optionally containing one double bond;
—(CH₂)_{w}—R₇ where R₇ is C₁-C₃ alkyl and C₁-C₂ alkoxy and where w is as defined above;

where R₃ and R₄ are taken together with the attached nitrogen atom to form a piperidinyl, piperazinyl, morpholinyl, pyrrolidinyl and pyridinyl ring;

and pharmaceutically acceptable salts thereof.

In another embodiment, the agent for treating an autophagy-related disorder may be a small organic molecule, such as a pyrazole of the formula:

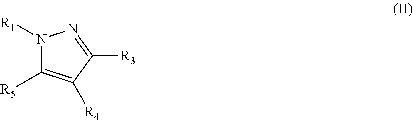

where R₁ is H—, CH₃—, C₂H₅— and cyclo C₃H₅—;

where R₃ is H—, F—, Cl—, Br—, I—, —NO₂, R₃₋₁-phenyl-CO—NH— where R₃₋₁ is CH₃—CO—, CH₃—, C₂H₅—, F—, Cl— and —NO₂;

where R₄ is H—, F—, Cl—, Br—, I—, —NO₂, —CO—O⁻, R₄₋₁-phenyl-CO—NH— where R₄₋₁ is CH₃—CO—, CH₃—, C₂H₅—, F—, Cl— and —NO₂;

where R₅ is H—, F—, Cl—, Br—, I—, —NO₂, R₅₋₁-phenyl-CO—NH— where R₃₋₁ is CH₃—CO—, CH₃—, C₂H₅—, F—, Cl— and —NO₂;

with the proviso:
(1) that one of R₃, R₄ and R₅ must be R₃₋₁-phenyl-CO—NH—, R₄₋₁-phenyl-CO—NH— or R₅₋₁-phenyl-CO—NH—;
   and pharmaceutically acceptable salts thereof.

In yet another embodiment the agent for treating an autophagy-related disorder may be a small organic molecule, such as a ketoester of the formula:

X₁—CO—O—CHR₁—CO—R₂ (III)

where X₁ is fluoren-9-one;

where R₁ is:
H—,
C₁-C₃ alkyl,
phenyl optionally substituted with one or two
   F—,
   Cl,
   —NO₂;

where R₂ is:
1-naphthyl,
2-naphthyl,
phenyl optionally substituted with one or two
   C₁-C₃ alkyl,
   C₁-C₂ alkoxy,
   F—,
   Cl—,
   Br—,
   —NO₂,
   —O—CO-phenyl optionally substituted with 1 F—, Cl— and CH₃—;
      and pharmaceutically acceptable salts thereof.

The agent for treating an autophagy-related disorder also may be a small organic molecule, such as a substituted phenyl compound of the formula:

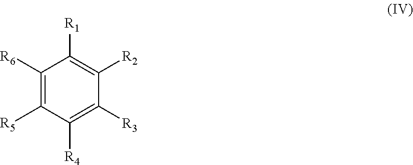

where R₁ is
   —CO—CH₃
   —CO—NH—R₁₋₁ where R₁₋₁ is
      naphthyl
      phenyl optionally substituted with one
         CH₃—CO—
         CH₃—CO—NH—
         phenyl-CO—CH═CH—
         Br—
         Cl—
         ⁻O—CO—;

where R₂ is —H, C₁-C₂ alkyl, —(CH₂)ₘ-phenyl where m is 1 or 2;

and where R₂ and R₃ are taken together with the atoms to which they are attached for form a phenyl ring optionally substituted with one —Cl, —Br and —CH₃;
where R₃ is —H, C₁-C₂ alkyl, —NO₂,
   —CO—NH-phenyl-CO—CH₃,
   —NH—CO—R₃₋₁ where R₃₋₁ is
      phenyl optionally substituted with —O—CO—CH₃,
      C₁-C₃ alkyl,
      2-furanyl,
phthalimide,
coumarin,
—O—CH₂-phenyl optionally substituted with one Cl—, Br— and CH₃—,
—SO₂—NR₃₋₂R₃₋₃ where R₃₋₂ is
   —H,
   C₁-C₃ alkyl and where R₃₋₃ is
   C₁-C₃ alkyl,
   phenyl optionally substituted with one C₁-C₂ alkyl,
   morpholinyl,
   piperidinyl,
   piperazinyl,
and where R₃ and R₄ are taken together with the atoms to which they are attached and —O—CH₂—O— to form a methylene dioxo ring;

where R₄ is H—, Cl—, Br— and C₁-C₂ alkyl;

and where R₄ and R₃ are taken together with the atoms to which they are attached and —O—CH₂—O— to form a methylene dioxo ring;
where R₅ is H—, C₁-C₂ alkyl, —NH—CO-phenyl, —NH—CO-phenyl-CO—CH₃ and —NH—CO—(C₁-C₂ alkyl);
where R₆ is H— and Cl—;
   and pharmaceutically acceptable salts thereof.

Also, the present invention includes a method of modulating autophagy in a cell, comprising administering to a cell an agent which modulates expression of PTPRS or PTPsigma, or which modulates the biological activity of PTPsigma; whereby autophagy in the cell is modulated. In further embodiments of this invention: the agent may be an antagonist or an agonist; the biological activity which is modulated may be the phosphatase activity of PTPsigma; or the agent may disrupt the interaction between PTPsigma and phosphatidylinositol 3-phosphate [PI(3)P] or phosphotyrosine (p-Tyr) protein. This inventive method also may be directed against autophagy-related disorders that may include a neurodegenerative disorder, an auto-immune disorder, a cardiovascular disorder, a metabolic disorder, hamartoma syndrome, a genetic muscle disorder, a myopathy, and a cancer. Further, agents that may be used in implementing the present invention include an inhibitory nucleic acid, a small organic molecule, an anti-PTPsigma antibody or antigen-binding fragment thereof, and derivatives thereof. In one embodiment, the agent may be an inhibitory nucleic acid selected from the group consisting of a siRNA targeting any one of the nucleic acids SEQ ID NOs: 3-7.

In another embodiment, the agent for modulating expression of PTPRS or PTPsigma, or for modulating the biological activity of PTPsigma may be a small organic molecule. One example of such an agent is a sulfonamide of the formula:

R₁—NH—SO₂—R₂—O—(CH₂)ₙ—CO—NR₃R₄ (I)

where n is 1 thru 3;

where R₁ is:
C₁-C₄ alkyl;
C₃-C₇ cycloalkyl;
phenyl-(CH₂)ₘ— where m is 0 thru 2 and phenyl is optionally substituted with one or two CH₃—, C₂H₅—, F— and Cl—;
phenyl-CH(CH₃)— where phenyl is optionally substituted with CH₃—, C₂H₅—, F— and Cl—;

where R₂ is phenyl optionally substituted with one F—, Cl—, CH₃—, C₂H₅—, and (CH₃)₂CH—;

where R₃ is H—:

where R₄ is:
C₁-C₃ alkyl;
C₃-C₇ cycloalkyl;
—CH₂—CH═CH₂
—(CH₂)_{z}—O—R₅ where z is 1 thru 5 and R₅ is C₁-C₃ alkyl;
—(CH₂)_{w}—R₆ where w is 1 thru 3 and R₆ is tetrahydrofuran or C₃-C₇ cycloalkyl optionally containing one double bond;
—(CH₂)_{w}—R₇ where R₇ is C₁-C₃ alkyl and C₁-C₂ alkoxy and where w is as defined above;

where R₃ and R₄ are taken together with the attached nitrogen atom to form a piperidinyl, piperazinyl, morpholinyl, pyrrolidinyl and pyridinyl ring;

and pharmaceutically acceptable salts thereof.

In another embodiment, the agent for modulating expression of PTPRS or PTPsigma, or for modulating the biological activity of PTPsigma may be a small organic molecule, such as a pyrazole of the formula:

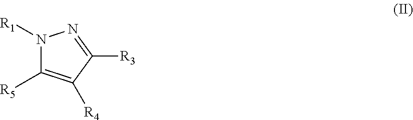

where R₁ is H—, CH₃—, C₂H₅— and cyclo C₃H₅—;

where R₃ is H—, F—, Cl—, Br—, I—, —NO₂, R₃₋₁-phenyl-CO—NH— where R₃₋₁ is CH₃—CO—, CH₃—, C₂H₅—, F—, Cl— and —NO₂;

where R₄ is H—, F—, Cl—, Br—, I—, —NO₂, —CO—O⁻, R₄₋₁-phenyl-CO—NH— where R₄₋₁ is CH₃—CO—, CH₃—, C₂H₅—, F—, Cl— and —NO₂;

where R₅ is H—, F—, Cl—, Br—, I—, —NO₂, R₅₋₁-phenyl-CO—NH— where R₃₋₁ is CH₃—CO—, CH₃—, C₂H₅—, F—, Cl— and —NO₂;

with the proviso:
(1) that one of R₃, R₄ and R₅ must be R₃₋₁-phenyl-CO—NH—, R₄₋₁-phenyl-CO—NH— or R₅₋₁-phenyl-CO—NH—;
   and pharmaceutically acceptable salts thereof.

In yet embodiment the agent for modulating expression of PTPRS or PTPsigma, or for modulating the biological activity of PTPsigma may be a small organic molecule, such as a ketoester of the formula:

X₁—CO—O—CHR₁—CO—R₂ (III)

where X₁ is fluoren-9-one;

where R₁ is:
H—,
C₁-C₃ alkyl,
phenyl optionally substituted with one or two
   F—,
   Cl,
   —NO₂;

where R₂ is:
1-naphthyl,
2-naphthyl,
   phenyl optionally substituted with one or two
      C₁-C₃ alkyl,
      C₁-C₂ alkoxy,
      F—,
      Cl—,
      Br—,
      —NO₂,
      —O—CO-phenyl optionally substituted with 1 F—, Cl— and CH₃—;
         and pharmaceutically acceptable salts thereof.

The agent for modulating expression of PTPRS or PTPsigma, or for modulating the biological activity of PTPsigma also may be a small organic molecule, such as a substituted phenyl compound of the formula:

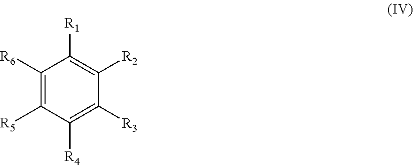

where R₁ is
   —CO—CH₃
   —CO—NH—R₁₋₁ where R₁₋₁ is
      naphthyl
      phenyl optionally substituted with one
         CH₃—CO—
         CH₃—CO—NH—
         phenyl-CO—CH═CH—
         Br—
         Cl—
         ⁻O—CO—;

where R₂ is —H, C₁-C₂ alkyl, —(CH₂)ₘ-phenyl where m is 1 or 2;

and where R₂ and R₃ are taken together with the atoms to which they are attached for form a phenyl ring optionally substituted with one —Cl, —Br and —CH₃;
where R₃ is —H, C₁-C₂ alkyl, —NO₂,
   —CO—NH-phenyl-CO—CH₃,
   —NH—CO—R₃₋₁ where R₃₋₁ is
      phenyl optionally substituted with —O—CO—CH₃,
      C₁-C₃ alkyl,
      2-furanyl,
phthalimide,
coumarin,
—O—CH₂-phenyl optionally substituted with one Cl—, Br— and CH₃—,
—SO₂—NR₃₋₂R₃₋₃ where R₃₋₂ is
   —H,
   C₁-C₃ alkyl and where R₃₋₃ is
   C₁-C₃ alkyl,
   phenyl optionally substituted with one C₁-C₂ alkyl,
   morpholinyl,
   piperidinyl,
   piperazinyl,
and where R₃ and R₄ are taken together with the atoms to which they are attached and —O—CH₂—O— to form a methylene dioxo ring;

where R₄ is H—, Cl—, Br— and C₁-C₂ alkyl;

and where R₄ and R₃ are taken together with the atoms to which they are attached and —O—CH₂—O— to form a methylene dioxo ring;
where R₅ is H—, C₁-C₂ alkyl, —NH—CO-phenyl, —NH—CO-phenyl-CO—CH₃ and —NH—CO—(C₁-C₂ alkyl);
where R₆ is H— and Cl—;
   and pharmaceutically acceptable salts thereof.

The present invention also includes a method for identifying an agent capable of modulating autophagy in a cell, comprising (a) providing (i) a PTPsigma polypeptide, or a PTPsigma homolog capable of binding to PI(3)P, and (ii) a test compound for screening; (b) mixing, in any order, the PTPsigma polypeptide, or the homolog, and the test compound; and (c) measuring the biological activity of the PTPsigma polypeptide, or the homolog, in the presence of the test compound as compared to the biological activity of the PTPsigma polypeptide, or the homolog, in the absence of the test compound; wherein a change in the biological activity of the PTPsigma polypeptide, or the homolog, in the presence of the test compound as compared to the absence of the test compound is indicative of a test compound that is an agent capable of modulating autophagy in a cell. In one embodiment of this inventive method, the test compound may be an inhibitory nucleic acid, a small organic molecule, an anti-PTP sigma antibody or antigen-binding fragment thereof, and derivatives thereof. In a further embodiment, the biological activity that is measured may the phosphatase activity of PTPsigma or the homolog.

Additionally, the present invention includes a method for identifying a test compound that modulates autophagy comprising (a) providing (i) a cell comprising a nucleic acid, or a fragment thereof, that encodes PTPsigma, or a PTPsigma homolog capable of binding to PI(3)P, and (ii) a test compound; (b) contacting the test compound and the cell; and (c) measuring the expression of the PTPsigma protein, or the homolog, in the cell in the presence of the test compound as compared to the expression of the PTPsigma protein, or homolog, in the cell in the absence of the test compound; wherein a change in expression of the PTPsigma protein, or homolog, in the cell in the presence of the test compound is indicative of a test compound that modulates autophagy. In further embodiments, the method may include an additional step of testing for autophagy; and the test compound may increase or decrease autophagy in the cell.

The present invention further includes a method of determining whether a subject is suffering from or is at risk for an autophagy-related disorder, including: (a) providing a biological sample obtained from a subject; and (b) determining whether the level of expression of PTPRS nucleic acid or PTPsigma polypeptide in the biological sample differs from the PTPRS or PTPsigma level of expression in a comparable biological sample obtained from a healthy subject.

The present invention also includes a pharmaceutical composition comprising an effective amount of an agent capable of modulating the expression of PTPRS or PTPsigma, or modulating the biological activity of PTPsigma, and a pharmaceutically acceptable carrier. In one embodiment the agent is an inhibitory nucleic acid; and the agent may be an siRNA targeting any one of the nucleic acids of SEQ ID NOs: 3-7.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIGS. 1A-1K show the results of a cell-based siRNA screen that identified PTPsigma as a modulator of PI(3)P. FIGS. 1A-1F: U2OS-2xFYVE-EGFP cells transfected with control siRNAs (a), VSP34 siRNAs (b), or starved of amino acids (c) were fixed and visualized by fluorescent microscopy (green: PI(3)P, 2xFYVE-EGFP; blue: nuclei, Hoechst). siRNAs targeting human phosphatase genes were screened to identify genes whose knockdown altered 2xFYVE-EGFP signal and distribution. Cells transfected with siRNAs targeting PTPRS (d), PTPN13 (e), and MTMR6 (f) are shown. FIG. 1G: Following knockdown of phosphatases, 2xFYVE-EGFP-positive punctae was qualitatively scored from −1 (decreased from control cells) to +1 (increased) and plotted. Phosphatases whose loss significantly increased 2xFYVE-EGFP fluorescence are highlighted in blue. FIG. 1H: Phospholipids were radiolabeled in vivo, extracted, and resolved by thin layer chromatography following transfection with control or PTPRS siRNAs. A PI(3)P standard was generated by incubating synthetic PtdIns with immunoprecipitated PI3K (p110/p85) and ³²P-ATP. The intensity of PI(3)P signal was measured by phosphorimaging and plotted. FIGS. 1I-1J: Endosomes were labeled by immunostaining with anti-EEA1 antibodies (FIG. 1I) and autophagic vesicles were labeled with anti-LC3B (FIG. 1J) antibodies following transfection with control or PTPRS siRNAs (red: EEA1 (FIG. 1I) or LC3B (FIG. 1J), rabbit-IgG-AF-546; blue: nuclei, Hoechst). FIG. 1K: Simplified model of PI(3)P regulation. Vps34 generates PI(3)P from PtdIns on endosomal and autophagic vesicles. Several myotubularin-related proteins (MTMs) have been shown to dephosphorylate PI(3)P and regulate endocytosis; here, we show PTPsigma controls PI(3)P and down-regulates autophagy.

FIGS. 2A-2I show that PTPsigma negatively regulates autophagy. FIGS. 2A-2F: U2OS cells transfected with control (FIG. 2A, 2C) or PTPRS siRNAs (FIG. 2D-2F) were cultured for 1 hr with full growth medium (FIG. 2A, 2D), 25 uM chloroquine (FIG. 2B, 2E), or 50 nM rapamycin and 25 uM chloroquine (FIG. 2C, 2F). Cells were stained with anti-LC3B antibodies and imaged by fluorescent microscopy (LC3B: pseudo-red, rabbit-IgG-AF488; nuclei: blue, Hoechst). FIG. 2G: LC3-I and LC3-II were analyzed by western blot using whole cell lysates from control siRNA-transfected cells, PTPRS siRNA-transfected cells, or amino-acid starved autophagic cells. α-tubulin was included as a loading control. FIG. 2H: ATG12 aggregates on autophagic structures were quantified by fluorescent microscopy using ATG12 immunostaining of control and PTPRS siRNA-transfected cells. Values plotted represent relative ATG12-positive AVs per cell following quantification of >75 cells and normalization to control cells cultured with nutrients. Bars represent standard error. FIG. 2I: V5-tagged PTPRS-CTF (BC104812; aa1156-1501) was transiently expressed in U2OS-2xFYVE-EGFP cells and PI(3)P and PTPRS colocalized (overlay) by confocal microscopy following 1 hr amino acid starvation (PI(3)P: green, 2xFYVE-EGFP; V5-PTPRS-CTF: red, rabbit-IgG-AF546; nuclei: blue, Hoechst).

FIG. 3A-3G show U2OS cells lacking PTPsigma and Ptprs−/− MEFs contain increased autophagic vesicles as identified by electron microscopy. FIGS. 3A-3D: Few double-membrane autophagic vesicles (AVs) were found by transmission electron microscopy (TEM) within control cells cultured in full nutrients (FIG. 3A), but were abundant in chloroquine-treated (FIG. 3B), amino acid (AA)-starved (FIG. 3C), and PTPRS siRNA-transfected (FIG. 3D) cells. Black arrows indicate autophagic vesicles. White arrowheads highlight double-membranes. FIGS. 3E-3G: Primary wild-type (Ptprs^{+/+}, FIG. 3E) and knockout (Ptprs^{-/-}, FIG. 3F) MEFs were analyzed by TEM and quantified (FIG. 3G). AVs, defined as double-membrane structures containing cytosolic components, were counted from ˜8.5 um2 sampling regions from two cells per type. Number of sampling areas (n) quantified is indicated. Bars represent standard error.

FIGS. 4A-4D show PTPsigma binds and dephosphorylates PI(3)P in vitro. FIG. 4A: GST-tagged recombinant enzymes (PTPRS-CTF; full-length MTMR6 and PTP1B) were incubated with water-soluble PI(3)P or phosphotyrosine peptide (p-Tyr) at 37° C. for 0.5 hr, released phosphates detected by malachite green binding, and absorbance measured at 650 nm. Phosphatase activity is expressed as percent activity compared to that with known substrate. FIG. 4B: The D1 domain of PTPsigma binds PI(3)P owing to a deep and wide active site cleft. Surface resonance of the active site is displayed (left panel). Negatively (red) and positively (blue) charged residues are shown and the PI(3)P molecule is drawn in ball-and-stick form. An active site cross-section is shown with bound PI(3)P (right panel). FIG. 4C: The crystal structure of the PTPRS D1 active site (PDB 2fh7) allows docking of PI(3)P with key residues highlighted. FIG. 4D: MTMR2 (PDB 1zsq) also binds PI(3)P with surface resonance and cross-sections indicated. All structures drawn with MolSoft ICM software.

FIGS. 5A-5D show PTPRS knockdown and amino acid starvation increase the abundance of cellular PI(3)P-positive vesicles. FIGS. 5A-5C: U2OS-2xFYVE-EGFP cells were transfected with control siRNA (FIG. 5A), PTPRS siRNA (FIG. 5B), or amino acid starved for 1 hr to induce autophagy (FIG. 5C). Cells were fixed, nuclei stained with Hoechst, and imaged by fluorescent microscopy (green: PI(3)P, 2xFYVE-EGFP). FIG. 5D: 2xFYVE-EGFP punctae were quantified using image analysis software (Imagine) from the field of cells shown. Mean 2xFYVE-EGFP-positive vesicles (punctae) per cell were determined and plotted (n=8, control; n=4, PTPRS siRNA; n=4, AA-starvation). Error bars represent standard deviation of 2xFYVE-EGFP-positive vesicles per cell.

FIGS. 6A-6J show target genes are effectively knocked down by siRNA. FIG. 6A: PTPN13 mRNA expression was depleted by 98% following transfection with PTPN13 siRNA for 48 hr. RNA extracted from control- or PTPN13-siRNA treated U2OS-2xFYVE-EGFP cells was converted to cDNA and PTPN13 levels determined by qRT-PCR using gene-specific primers. Values were normalized to GAPDH. FIG. 6B: MTMR6 mRNA expression was depleted by 89% following siRNA transfection as determined by the methods above. FIG. 6C: Western blot analysis of whole cell lysates following transfection with control or VPS34 siRNA showing depletion of VPS34 protein levels. α-tubulin was analyzed as a loading control. FIGS. 6D-6I: U2OS-2xFYVE-EGFP cells were transfected with control (FIG. 6D) or PTPRS siRNA (FIG. 6E, siRNA-A; FIG. 6F, siRNA-B; FIG. 6G, siRNA-C; FIG. 6H, siRNA-D; FIG. 6I, siRNA-pool (FIGS. 6A-6D)) for 48 hr, fixed, and imaged by fluorescent microscopy (PI(3)P: green, 2xFYVE-EGFP; nuclei: blue, Hoechst). FIG. 6J, PTPRS mRNA knockdown following 48 hr siRNA transfection was determined by qRT-PCR using gene-specific primers and GAPDH normalization as outlined above.

FIGS. 7A-7B show PTPsigma overexpression reduces cellular PI(3)P. FIG. 7A: U2OS-2xFYVE-EGFP cells were transfected with V5-PTPRS-CTF (BC104812; aa1156-1501) for 24 hrs, fixed, and imaged by fluorescence microscopy (PI(3)P: green, 2xFYVE-EGFP; PTPRS: red, mouse-IgG-AF546; nuclei: blue, Hoechst). White arrows indicate PTPRS-transfected cells. FIG. 7B: Prior to fixation, cells were starved of amino acids for 1 hr to induce autophagy and imaged as described above. White arrows indicate PTPRS-transfected cells.

FIG. 8 shows siRNA-mediated knockdown of human phosphatase genes alters cellular PI(3)P. U2OS-2xFYVE-EGFP cells were transfected with siRNA targeting human phosphatase genes for 48 hrs (4 siRNA sequences per gene per well). Following knockdown, 2xFYVE-EGFP signal and distribution was visualized by confocal microscopy and qualitatively scored from −1 (decreased punctae from control cells) to +1 (increased punctae). Knockdown of each gene was performed in triplicate and each replicate was independently scored by two individuals. The sum scores for each gene are displayed for scorer 1 (column 4) and scorer 2 (column 5). The mean score was calculated (column 8) by dividing the total score (column 6) by the possible score (column 7). Gene symbols are displayed (column 1) as well as plate (column 2) and well (column 3) position of transfection.

FIGS. 9A and 9B show small molecules decrease PTPsigma phosphatase activity in vitro. In particular, FIG. 9A shows the chemical structures of PTPsigma small molecule inhibitors RS-6, RS-46, RS-48, RS-49. FIG. 9B shows the inhibition PTPRS activity by these small molecule inhibitors.

FIG. 10 shows the chemical structures of nineteen PTPsigma small molecule inhibitors.

FIG. 11 shows the inhibition of PTPRS activity by the small molecule inhibitors shown in FIG. 10.

FIGS. 12-15 show the chemical structures of various small molecule inhibitors which are expected to inhibit PTPRS activity.

### DETAILED DESCRIPTION OF THE PREFERRED EMBODIMENTS

The details of one or more embodiments of the invention are set forth in the accompanying drawings and the description below. Other features, embodiments, and advantages of the invention will be apparent from the description, drawings, examples, the Sequence Listing and from the claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. In the case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the worldwide web at ncbi.nlm.nih.gov.

The information provided herein is not admitted to be prior art to the present invention, but is provided solely to assist the understanding of the reader.

For clarity of disclosure, and not by way of limitation, the detailed description of the invention is divided into the subsections that follow.

Definitions

Unless defined otherwise, all technical and scientific terms used herein have the meaning commonly understood by one of ordinary skill in the art to which this invention belongs. Generally, the nomenclature used herein and the practices of the present invention described herein are techniques in cell biology, cell culture, molecular biology, transgenic biology, microbiology, recombinant DNA, immunology, organic chemistry and nucleic acid chemistry, and are well known and commonly employed in the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); MuIHs et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. I. Freshney, ed. (1987)), Manipulating the Mouse Embryo, (Cold Spring Harbor Laboratory Press, Cold Spring Harbor, N.Y., 1986). Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

The term “a 1 to 6 nucleotide overhang on at least one of the 5′ end or 3′ end” as used herein means the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The term “agent” is used herein to mean all materials that may be used to prepare pharmaceutical and diagnostic compositions, or that may be a chemical compound, a mixture of chemical compounds, a biological macromolecule (such as a nucleic acid, an antibody or fragment thereof, a protein or portion thereof, e.g., a peptide), an extract made from biological materials such as bacteria, plants, fungi, or animal (particularly mammalian) cells or tissues, or a fragment, isoform, variant, derivative, or other material that may be used independently for such purposes, all in accordance with the present invention. The activity of such agents may render it suitable as a “therapeutic agent” which is a biologically, physiologically, or pharmacologically active substance (or substances) that acts locally or systemically in a subject.

The terms “agonist” or “activator” are used herein to mean an agent that upregulates (e.g., activates or enhances) at least one biological activity of a protein. For example, an agent is an agonist of the phosphatase PTPsigma if the agent upregulates the phosphatase activity PTPsigma on PI(3)P or p-Tyr protein. An agonist may be a compound which increases the interaction between a protein and another molecule, e.g., a target peptide or enzyme substrate. An agonist may also be a compound that increases expression of a gene or which increases the amount of protein expressed.

The terms “antagonist” or “inhibitor” are used herein to mean an agent that downregulates (e.g., suppresses or inhibits) at least one bioactivity of a protein. For example, an agent is an antagonist of the phosphatase PTPsigma if the agent downregulates the phosphatase activity PTPsigma on PI(3)P. An antagonist may be a compound which inhibits or decreases the interaction between a protein and another molecule, e.g., a target peptide or enzyme substrate. An antagonist may also be a compound that downregulates expression of a gene or which reduces the amount of protein expressed.

The term “antibody” as used herein means a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology.

The term “antigen-binding fragment” as used herein means (i) a Fab fragment, a monovalent fragment consisting of the V_{L}, V_{H}, C_{L} and C_{H1} domains, (ii) a F(ab′)₂ fragment, a bivalent fragment comprising two Fab fragments linked by a disulfide bridge at the hinge region, (iii) a Fd fragment consisting of the V_{H} and C_{H1} domains, (iv) a Fv fragment consisting of the V_{L} and V_{H} domains of a single arm of an antibody, (v) a dAb fragment (Ward et al (1989) Nature 341 544-46), which consists of a VH domain, and (vi) an isolated complementarity determining region (CDR). Camelid antibodies, and camelized antibodies can also be used. Such antibodies, e.g., can include CDRs from just one of the variable domains of the antibody. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv), see, e.g., Bird et al (1988) Science 242 423-26, Huston et al (1988) Proc Natl Acad Sci USA 85 5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

The term “antisense strand” as used herein means the strand of a siRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “autoimmune disorders” as used herein means, but is not limited to, rheumatoid arthritis, Graves' disease, multiple sclerosis, scleroderma, autoimmune hepatitis, fibromyalgia, myasthenia gravis (MG), systemic lupus erythematosis (SLE), graft rejection (e.g., allograft rejection), and T cell disorders (including acquired immune deficiency syndrome (AIDS)).

The term “autophagy” is used herein to mean a catabolic process involving the degradation of a cell's own components through the lysosomal machinery. It is a tightly-regulated process that plays a part in normal cell growth, development, and homeostasis, helping to maintain a balance between the synthesis, degradation, and subsequent recycling of cellular products. A variety of autophagic processes exist, all having in common the degradation of intracellular components via the lysosome. The most well-known mechanism of autophagy involves the formation of a membrane around a targeted region of the cell, separating the contents from the remainder of the cytoplasm; the resultant vesicle then fuses with a lysosome and subsequently degrades the contents.

The term “autophagy-related disorders” as used herein means a disorder that is caused by associated with, the result of, or otherwise related to aberrant autophagy and this term includes, but is not limited to, cancers, cardiovascular disorders, neurodegenerative disorders, and autoimmune disorders, metabolic disorders, hamartoma syndrome, genetic muscle disorders, and myopathies.

The term “binding” is used herein to mean an association, which may be a stable association, between two molecules [e.g., between PTPsigma and PI(3)P or p-Tyr protein] due to, for example, electrostatic, hydrophobic, ionic and/or hydrogen-bond interactions under physiological conditions.

The term “biological sample” as used herein means sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The term “cancer” as used herein means solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

The term “cardiovascular disorders” as used herein means, but is not limited to stroke, acute coronary syndromes including unstable angina, thrombosis and myocardial infarction; atherosclerosis (or arteriosclerosis); plaque rupture; both primary and secondary (in-stent) restenosis in coronary or peripheral arteries; transplantation-induced sclerosis; peripheral limb disease; ischemic heart disease (e.g., angina pectoris, myocardial infarction, and chronic ischemic heart disease); hypertensive heart disease; pulmonary heart disease; valvular heart disease (e.g., rheumatic fever and rheumatic heart disease, endocarditis, mitral valve prolapse, and aortic valve stenosis); preeclampsia; peripheral vascular disease; atrial or ventricular septal defect; myocardial disease (e.g., myocarditis, myocardial ischemia, congestive cardiomyopathy, and hypertrophic cariomyopathy); and diabetic complications (including ischemic heart disease, peripheral artery disease, congestive heart failure, retinopathy, neuropathy and nephropathy).

The term “carrier” refers to a diluent, adjuvant, excipient, or vehicle with which the compound is administered. Carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “complementary” as used herein to describe a first nucleotide sequence in relation to a second nucleotide sequence, unless otherwise stated, means the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides. This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a siRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

The terms “complementary”, “fully complementary” and “substantially complementary” are used herein with respect to the base matching between the sense strand and the antisense strand of a siRNA, or between the antisense strand of a siRNA and a target sequence, as will be understood from the context of their use.

The term “complementary sequences” as used herein, means a nucleic acid including, or formed entirely from non-Watson-Crick base pairs and/or base pairs formed from non-natural and modified nucleotides, in as far as the above requirements with respect to their ability to hybridize are fulfilled.

The term “cure” as used herein means to lead to the remission of the disorder associated with autophagy in a subject, or of ongoing episodes thereof, through treatment.

The term “delay of progression” as used herein means that the administration of an agent or pharmaceutical composition to subjects in a pre-stage or in an early phase of a disorder (e.g., a associated with aberrant autophagy in a subject (e.g., an autoimmune disorders)) prevents the disease from evolving further, or slows down the evolution of the disease in comparison to the evolution of the disease without administration of the pharmaceutical composition.

The terms “derivative” or “derivatives” as used herein means either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

The term “double-stranded RNA” or “dsRNA”, as used herein means a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where they are separate RNA molecules, such siRNA are often referred to in the literature as siRNA (“short interfering RNA”). Where the two strands are different portions of one larger RNA molecule, and therefore are connected by an uninterrupted chain of nucleotides between the 3′-end of one strand and the 5′ end of the respective other strand forming the duplex structure, the connecting RNA chain is referred to as a “hairpin loop”, “short hairpin RNA” or “shRNA”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the siRNA minus any overhangs that are present in the duplex. In addition to the duplex structure, a siRNA may comprise one or more nucleotide overhangs. In addition, as used in this specification, “siRNA” may include chemical modifications to ribonucleotides, including substantial modifications at multiple nucleotides and including all types of modifications disclosed herein or known in the art. Any such modifications, as used in an siRNA type molecule, are encompassed by “siRNA” for the purposes of this specification and claims.

The term “each strand is 49 nucleotides or less” as used herein means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The terms “effective amount” and “therapeutically effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

The term “inhibit the expression of”, referring to the PTPRS gene, as used herein means the at least partial suppression of the expression of the PTPRS gene as manifested by a reduction of the amount of mRNA transcribed from the PTPRS gene which may be isolated from a first cell or group of cells in which the PTPRS gene is transcribed and which has or have been treated such that the expression of the PTPRS gene is inhibited, as compared to a second cell or group of cells substantially identical to the first cell or group of cells but which has or have not been so treated (control cells). The degree of inhibition is usually expressed in terms of (mRNA in control cells)−(mRNA in treated cells) divided by (mRNA in control cells) multiplied by 100 percent. Alternatively, the degree of inhibition may be given in terms of a reduction of a parameter that is functionally linked to PTPRS gene transcription, e.g. the amount of PTPsigma protein encoded by the PTPRS gene, or the number of cells displaying a certain phenotype. In principle, inhibiting expression of the PTPRS gene may be determined in any cell expressing the target, either constitutively or by genomic engineering, and by any appropriate assay. In certain instances, expression of the PTPRS gene is suppressed by at least about 5%, 10%, 20%, 25%, 35%, or 50% by administration of the agent of the present invention. In some embodiments, the PTPRS gene is suppressed by at least about 60%, 70%, or 80% by administration of the agent. In some embodiments, the PTPRS gene is suppressed by at least about 85%, 90%, 95%, or 99% by administration of the agent.

The term “inhibitory nucleic acid” as used herein means nucleic acid compounds capable of producing gene-specific inhibition of gene expression. Typical inhibitory nucleic acids include, but are not limited to, antisense oligonucleotides, triple helix DNA, RNA aptamers, ribozymes and short inhibitory RNAs (“siRNAs”). For example, knowledge of a nucleotide sequence may be used to design siRNA or antisense molecules which potently inhibit the expression of PTPRS. Similarly, ribozymes can be synthesized to recognize specific nucleotide sequences of a gene and cleave it. Techniques for the design of such molecules for use in targeted inhibition of gene expression are well known to one of skill in the art.

The term “introducing into a cell” when used herein to refer to a siRNA means facilitating uptake or absorption into the cell, as is understood by those skilled in the art. Absorption or uptake of siRNA can occur through unaided diffusive or active cellular processes, or by auxiliary agents or devices. The meaning of this term is not limited to cells in vitro; a siRNA may also be “introduced into a cell”, wherein the cell is part of a living organism. In such instance, introduction into the cell will include the delivery to the organism. For example, for in vivo delivery, siRNA can be injected into a tissue site or administered systemically. In vitro introduction into a cell includes methods known in the art such as electroporation and lipofection.

The term “modulation” (and other formulations of this term, e.g., “modulate”, “modulates”, and “modulating”) when used herein in reference to a functional property or biological activity or process (e.g., phosphatase activity or receptor binding), means the capacity to control or influence directly or indirectly, and by way of non-limiting examples, can alternatively mean inhibit or stimulate, agonize or antagonize, hinder or promote, activate or suppress, and strengthen or weaken, or otherwise change a quality of such property, activity or process. The modulation is manifested by an increase or a decrease in the expression level of a gene or protein, or the level of a functional property or biological activity from a first cell, group of cells, subject, or subjects in which an agent has been administered as compared to the expression level, level of a functional property or biological activity in a second cell, group of cells, subject, or subjects in which the agent has not been administered (controls). The modulation described herein can be determined by any appropriate assay, such as those described herein below. In certain instances, the expression level of a gene or protein, or the level of a functional property or biological activity from the first cell, group of cells, subject, or subjects is increased or decreased by at least about 5%, 10%, 20%, 25%, 35%, or 50% by administration of the agent as compared to the second cell, group of cells, subject, or subjects. In some embodiments, the expression level of a gene or protein, or the level of a functional property or biological activity from the first cell, group of cells, subject, or subjects is increased or decreased by at least about 60%, 70%, or 80% by administration of the agent as compared to the second cell, group of cells, subject, or subjects. In some embodiments, the expression level of a gene or protein, or the level of a functional property or biological activity from the first cell, group of cells, subject, or subjects is increased or decreased by at least about 85%, 90%, 95%, or 99% by administration of the agent as compared to the second cell, group of cells, subject, or subjects.

The term “neurodegenerative disorders” as used herein means, but is not limited to, Huntington's disease, Parkinson's Disease, Alzheimer's Disease, dystonia, dementia, multiple sclerosis, Amyotrophic Lateral Sclerosis (ALS), and Creutzfeld-Jacob Disease.

The terms “normal mammalian cell” and “normal animal cell” as used herein mean cells that are growing under normal growth control mechanisms (e.g., genetic control) and display normal cellular differentiation. Cancer cells differ from normal cells in their growth patterns and in the nature of their cell surfaces. For example cancer cells tend to grow continuously and chaotically, without regard for their neighbors, among other characteristics well known in the art.

The term “nucleotide overhang” as used herein means the unpaired nucleotide or nucleotides that protrude from the duplex structure of a siRNA when a 3′-end of one strand of the siRNA extends beyond the 5′-end of the other strand, or vice versa. “Blunt” or “blunt end” means that there are no unpaired nucleotides at that end of the siRNA, i.e., no nucleotide overhang. A “blunt ended” siRNA is a siRNA that is double-stranded over its entire length, i.e., no nucleotide overhang at either end of the molecule. For clarity, chemical caps or non-nucleotide chemical moieties conjugated to the 3′ end or 5′ end of an siRNA are not considered in determining whether an siRNA has an overhang or is blunt ended.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “phosphatase” is used herein to mean an enzyme that removes a phosphate group from a substrate by hydrolysis. For example, as discovered by the inventors and described herein, PTPsigma is a phosphatase that remove a phosphate group from PI(3)P or p-Tyr protein.

The term “purified” as used herein means an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis, mass-spectrometry analysis.

The terms “prophylaxis” or “prevention” as used herein mean impeding the onset or recurrence of autophagy-related disorders, e.g., autoimmune disorders.

The term “sense strand” as used herein means the strand of a siRNA that includes a region that is substantially complementary to a region of the antisense strand.

The term “siRNA” is used herein to mean a short (or small) interfering RNA. siRNAs comprise two sequences that are essentially complementary to each other so that they can hybridize under the desired conditions. The two sequences may be present on one strand or on two strands of nucleic acid. For example, the two sequences may be on one nucleic acid and separated by a spacer sequence that may form a loop when the two sequences interact.

The term “small organic molecule,” or “small molecule,” as used herein means an organic compound (or organic compound complexed with an inorganic compound, e.g., metal) that has a molecular weight of less than 3 kilodaltons, and preferably less than 1.5 kilodaltons.

The term “strand comprising a sequence” as used herein means an oligonucleotide comprising a chain of nucleotides that is described by the sequence referred to using the standard nucleotide nomenclature.

As used herein the term “subject” refers to any multi-cellular living organism. In some embodiments, the subject is a mammal. The mammal can be any mammal including, but not limited to, mammals of the order Rodentia, such as mice and hamsters, and mammals of the order Logomorpha, such as rabbits. In some embodiments, the mammals are from the order Carnivora, including Felines (cats) and Canines (dogs). In some embodiments, the mammals are from the order Artiodactyla, including Bovines (cows) and Swines (pigs) or of the order Perssodactyla, including Equines (horses). In some embodiments, the mammals are of the order Primates, Ceboids, or Simoids (monkeys) or of the order Anthropoids (humans and apes). A preferred animal subject of the present invention is a mammal. The invention is particularly useful in the treatment of human subjects.

The term “substantially complementary to at least part of a mRNA” when used herein to refer to a polynucleotide means a polynucleotide which is substantially complementary to a contiguous portion of the mRNA of interest (e.g., an mRNA encoding PTPsigma). For example, a polynucleotide is complementary to at least a part of a PTPsigma mRNA if the sequence is substantially complementary to a non-interrupted portion of a mRNA encoding PTPsigma.

The term “suppress and/or reverse,” e.g., a disorder associated with autophagy in a subject (e.g., an autoimmune disease), is used herein to mean abrogating said condition, or rendering said condition less severe than before or without the treatment.

The term “target sequence” as used herein means a contiguous portion of the nucleotide sequence of an mRNA molecule formed during the transcription of the PTPRS gene, including mRNA that is a product of RNA processing of a primary transcription product.

The term “test compound” is used herein to mean a molecule to be tested by one or more screening method(s) as a putative agent that is capable of modulating: autophagy in a cell, expression of PTPRS or PTPsigma; the biological activity of PTPsigma; or other biological entity or process. The term “control test compound” refers to a compound known to bind to the target (e.g., a known agonist, antagonist, partial agonist or inverse agonist). The term “test compound” does not include a chemical added as a control condition that alters the function of the target to determine signal specificity in an assay. Such control chemicals or conditions include chemicals that 1) nonspecifically or substantially disrupt protein structure [e.g., denaturing agents (e.g., urea or guanidinium], chaotropic agents, sulfhydryl reagents (e.g., dithiothreitol and β-mercaptoethanol), and proteases), 2) generally inhibit cell metabolism (e.g., mitochondrial uncouplers) and 3) non-specifically disrupt electrostatic or hydrophobic interactions of a protein (e.g., high salt concentrations, or detergents at concentrations sufficient to non-specifically disrupt hydrophobic interactions). In certain embodiments, various predetermined concentrations of test compounds are used for screening such as 0.01 μM, 0.1 μM, 1.0 μM, and 10.0 μM. Examples of test compounds include, but are not limited to, antibodies and antigen-binding fragments thereof, peptides, nucleic acids, carbohydrates, and small organic molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay. Further, the activity of a test compound may render it suitable as a “therapeutic agent” which is a biologically, physiologically, or pharmacologically active substance (or substances) that acts locally or systemically in a subject. Thus, a therapeutic agent refers to any substance that intended for use in the diagnosis, cure, mitigation, treatment or prevention of disease or in the enhancement of desirable physical or mental development and/or conditions in an animal or human.

The term “treatment” (and other formulations of this term, e.g., “treat”, “treats”, and “treating”) as used herein means administering to a subject an agent or pharmaceutical composition (variant or chemical derivative). This term does not necessarily imply 100% or complete treatment. Rather, there are varying degrees of treatment of which one of ordinary skill in the art recognizes as having a potential benefit or therapeutic effect. In this respect, the inventive methods can provide any amount of any level of treatment of an autophagy-related disorder in a subject. Furthermore, the treatment provided by the inventive method can include treatment of one or more conditions or symptoms of the disease or condition being treated, and/or can include the retarding of the progression of the disease or condition. Treating also includes administering an agent to a subject at risk for developing an autophagy-related disorder prior to evidence of clinical disease, as well as subjects diagnosed with an autophagy-related disorder who have not yet been treated or who have been treated by other means. Thus, this invention is useful in preventing or inhibiting an autophagy-related disorder.

General

Through the use of a high-content cell-based RNAi screen, the inventors identified phosphatases whose knockdown elevates cellular PI(3)P. Notably, RNAi-mediated knockdown of MTMR6 resulted in swollen and often perinuclear PI(3)P-positive vesicles. Previous studies have shown similar phenotypes when endocytic PI(3)P is elevated, for example by constitutive activation of early endosomal Rab5, or knockdown of the PI5 kinase (PIKfyve). Accordingly, these PI(3)P-positive vesicles are endosomal and these phosphatases may function in endocytic signaling.

The present disclosure is based, at least in part, on the striking result from this study of the accumulation, following knockdown of PTPsigma, of abundant PI(3)P-positive vesicles, which phenocopies autophagic cells. The inventors have shown that PTPsigma harbors in vitro phosphatase activity against PI(3)P in addition to its function as a PTPase against p-Tyr peptides. The concept of classically defined PTPs using lipids as physiological substrates is not unprecedented: PTEN was originally identified as a protein tyrosine phosphatase. Importantly, the inventors show that PTPsigma is a PI(3)P phosphatase that selectively regulates autophagy. Comparative structural analysis of PTPsigma to a known phosphoinositide phosphatase, MTMR2, revealed their active site pockets to be similar in depth and width. The phosphatase active site pocket is uniquely shaped to not only bind a tyrosine or inositol ring, but to also be wide enough to accommodate the 1′ phosphate linking the phosphoinositol head group to the glycerol backbone and fatty acid chains. Although the overall depth is similar to that of PTP1B, the PTP1B pocket is narrower and excludes PI(3)P, binding only phosphotyrosine. For enzymes such as PTPsigma, the inventors refer to them as binary function phosphatases, reflecting the ability to dephosphorylate two different substrates, both phosphotyrosine and phosphoinositides.

Methods of Treating Auophagy-Related Disorders

Provided herein are methods for treating diseases that can benefit from modulation of the expression level of PTPRS or PTPsigma, or the activity level of PTPsigma. More specifically, the present invention includes a method of treating an autophagy-related disorder in a subject, comprising administering to the subject an effective amount of an agent which modulates expression of the gene encoding tyrosine phosphatase receptor type sigma (PTPRS) or the PTPRS gene product (PTPsigma), or which modulates the biological activity of the PTPRS gene product (PTPsigma).

An illustrative method comprises administering to a subject in need thereof a therapeutically effective amount of an agent capable of modulating the level of PTPRS or PTPsigma, or modulating the biological activity of PTPsigma. A method may comprise administering two or more agents. An agent may be any agent described herein or an agent identified by a screening method, e.g., those described herein. For example, an agent may be an siRNA or a small organic molecule that modulates the activity or protein level of PTPsigma.

Diseases that can be treated or prevented include those that are associated with abnormal autophagy. For example, diseases in which autophagy is desired can be treated with agents that induce autophagy, e.g., inhibitors of PTPsigma. Such diseases include those in which excessive cell proliferation occurs, such as those associated with the formation of tumors, e.g., cancer, warts, or other growths. Autoimmune diseases could also be targeted. Exemplary cancers that can be treated are further described herein.

Other diseases that can be treated or prevented include those in which defective autophagy occurs, such as neurodegenerative diseases. Such diseases can be treated or prevented with agents that activate autophagy, e.g., inhibitors of PTPsigma.

Modulating expression of PTPRS or PTPsigma in a subject may occur when the level of expression of PTPsigma is increased or decreased as compared to a control. Suitable controls are described herein and are otherwise known in the art. In certain instances, expression of the PTPRS gene or PTPsigma is increased or decreased by at least about 20%, 25%, 35%, or 50% by administration of an agent. In some embodiments, the PTPRS or PTPsigma is increased or decreased by at least about 60%, 70%, or 80% by administration of an agent. In some embodiments, the PTPRS or PTPsigma is increased or decreased by at least about 85%, 90%, or 95% by administration of an agent. The gene or protein expression, and therefore its modulation, can be measured as described herein or as otherwise known in the art.

Modulating the biological activity of PTPsigma occurs when the biological activity of PTPsigma is increased or decreased as compared to a control. Suitable controls are described herein and are otherwise known in the art. In certain instances, the biological activity of PTPsigma is increased or decreased by at least about 20%, 25%, 35%, or 50% by administration of an agent. In some embodiments, the biological activity of PTPsigma is increased or decreased by at least about 60%, 70%, or 80% by administration of an agent. In some embodiments, the modulation of PTPsigma is increased or decreased by at least about 85%, 90%, or 95% by administration of an agent. The biological activity which is modulated may be the phosphatase activity of PTPsigma as a PTPase or as an phosphatase that dephosphorylates PI(3)P or p-Tyr protein, which can be measured as described herein or as otherwise known in the art.

Further, in one embodiment, the agent disrupts the interaction between PTPsigma and phosphatidylinositol 3-phosphate [PI(3)P] or p-Tyr protein. This disruption of this interaction can be measured by various assays that are known in the art and/or are described herein.

Agents useful in the practice of the present method are capable of modulating the level of PTPRS or PTPsigma, or modulating the biological activity of PTPsigma. Such agents include an inhibitory nucleic acid, a small organic molecule, an anti-PTPsigma antibody or antigen-binding fragment thereof, and derivatives thereof.

In one embodiment of the invention, the agent, or component of the pharmaceutical composition, is an inhibitory nucleic acid, such as a small interfering ribonucleic acid (siRNA). siRNAs decrease or block gene expression. While not wishing to be bound by theory, it is generally thought that siRNAs inhibit gene expression by mediating sequence specific mRNA degradation. RNA interference (RNAi) is the process of sequence-specific, post-transcriptional gene silencing, particularly in animals and plants, initiated by double-stranded RNA (dsRNA) that is homologous in sequence to the silenced gene (Elbashir et al. Nature 2001; 411(6836): 494-8). Accordingly, it is understood that siRNAs and long dsRNAs having substantial sequence identity to all or a portion of a polynucleotide of the present invention may be used to inhibit the expression of a nucleic acid of the invention, and particularly when the polynucleotide is expressed in a mammalian or plant cell.

Alternatively, siRNAs that decrease or block the expression of the phosphatase described herein may be determined by testing a plurality of siRNA constructs against the target gene. Such siRNAs against a target gene may be chemically synthesized. The nucleotide sequences of the individual RNA strands are selected such that the strand has a region of complementarity to the target gene to be inhibited (i.e., the complementary RNA strand comprises a nucleotide sequence that is complementary to a region of an mRNA transcript that is formed during expression of the target gene, or its processing products, or a region of a (+) strand virus). The step of synthesizing the RNA strand may involve solid-phase synthesis, wherein individual nucleotides are joined end to end through the formation of internucleotide 3′-5′ phosphodiester bonds in consecutive synthesis cycles.

Various assays are known in the art to test siRNA for its ability to mediate RNAi (see for instance Elbashir et al., Methods 26 (2002), 199-213). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

Provided herein are siRNA molecules comprising a nucleotide sequence consisting essentially of a sequence of PTPRS. The use of these siRNAs enables the targeted degradation of mRNAs of PTPsigma. An siRNA molecule may comprise two strands, each strand comprising a nucleotide sequence that is at least essentially complementary to each other, one of which corresponds essentially to a sequence of a target gene. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

The sequence that corresponds essentially to a sequence of a target gene is referred to as the “sense target sequence” and the sequence that is essentially complementary thereto is referred to as the “antisense target sequence” of the siRNA. The length of the region of the siRNA complementary to the target, in accordance with the present invention, may be from about 10 to about 100 nucleotides, about 12 to about 25 nucleotides, about 14 to about 22 nucleotides or 15, 16, 17 or 18 nucleotides. Where there are mismatches to the corresponding target region, the length of the complementary region is generally required to be somewhat longer. Because the siRNA may carry overhanging ends (which may or may not be complementary to the target), or additional nucleotides complementary to itself but not the target gene, the total length of each separate strand of siRNA may be from about 10 to about 100 nucleotides, about 15 to about 49 nucleotides, about 17 to about 30 nucleotides or about 19 to about 25 nucleotides.

The length of the sense and antisense sequences is determined so that an siRNA having sense and antisense target sequences of that length is capable of inhibiting expression of a target gene, preferably without significantly inducing a host interferon response. Where there are mismatches to the corresponding target region, the length of the complementary region is generally required to be somewhat longer.

The sense and antisense target sequences are preferably sufficiently complimentary, such that an siRNA comprising both sequences is able to inhibit expression of the target gene, i.e., to mediate RNA interference. For example, the sequences may be sufficiently complementary to permit hybridization under the desired conditions, e.g., in a cell. Accordingly, the sense and antisense target sequences may be at least about 95%, 97%, 98%, 99% or 100% identical and may, e.g., differ in at most 5, 4, 3, 2, 1 or 0 nucleotides.

The siRNA molecules in accordance with the present invention may comprise a double-stranded region which is substantially identical to a region of the mRNA of PTPRS. A region with 100% identity to the corresponding sequence of the target gene is suitable. This state is referred to as “fully complementary.” However, the region may also contain one, two or three mismatches as compared to the corresponding region of the target gene, depending on the length of the region of the mRNA that is targeted, and as such may be not fully complementary. In an embodiment, the RNA molecules of the present invention specifically target PTPRS. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. The region of a target gene effective for inhibition by the RNAi reagent may be determined by experimentation. A suitable mRNA target region would be the coding region. Also suitable are untranslated regions, such as the 5′-UTR, the 3′-UTR, and splice junctions. Table 1 provides examples of target sequences (5′ to 3′) that can be utilized to implement various embodiments of the present invention.

TABLE 1

| | |
|---|---|
| CACGGCATCAGGCGTGCACAA | (SEQ ID NO. 3) |
| CGCGTCTACTACACCATGGAA | (SEQ ID NO. 4) |
| CAGGACATTCTCTCTGCACAA | (SEQ ID NO. 5) |
| AAGAACAAACCCGACAGTAAA | (SEQ ID NO. 6) |
| CACAGGCTGCTTTATCGTCAT | (SEQ ID NO. 7) |

The siRNA according to the present invention may confer a high in vivo stability suitable for oral delivery by including at least one modified nucleotide in at least one of the strands. Thus the siRNA according to the present invention may contain at least one modified or non-natural ribonucleotide. Suitable modifications for oral delivery include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates). Finally, end modifications sometimes referred to herein as 3′ caps or 5′ caps may be of significance. Caps may consist of more complex chemistries which are known to those skilled in the art.

In one embodiment, the invention provides double-stranded ribonucleic acid (dsRNA) molecules for inhibiting the expression of PTPRS. The dsRNA comprises at least two sequences that are complementary to each other. The dsRNA comprises a sense strand comprising a first sequence and an antisense strand comprising a second sequence. The antisense strand comprises a nucleotide sequence which is substantially complementary to at least part of an mRNA encoding PTPsigma, and the region of complementarity is less than 30 nucleotides in length, generally 19-24 nucleotides in length. The nucleotide sequences of the sense and antisense strands of exemplary siRNAs are provided in Table 2. Other siRNAs may comprise a sequence consisting essentially of the sequences disclosed in Table 2 with one or more, or one or less, nucleotides at one or both ends.

TABLE 2

| | |
|---|---|
| Sense strand targeting | SEQ ID NO. 8 |
| SEQ ID NO. 3: | |
| 5′-CGGCAUCAGGCGUGCACAATT | |
| Antisense strand targeting | SEQ ID NO. 9 |
| SEQ ID NO. 3: | |
| 5′-UUGUGCACGCCUGAUGCCGTG | |
| Sense strand targeting | SEQ ID NO. 10 |
| SEQ ID NO. 4: | |
| 5′-(CGUCUACUACACCAUGGAA)TT | |
| Antisense strand targeting | SEQ ID NO. 11 |
| SEQ ID NO. 4: | |
| 5′-(UUCCAUGGUGUAGUAGACG)TG | |
| Sense strand targeting | SEQ ID NO. 12 |
| SEQ ID NO. 5: | |
| 5′-GGACAUUCUCUCUGCACAATT | |
| Antisense strand targeting | SEQ ID NO. 13 |
| SEQ ID NO. 5: | |
| 5′-UUGUGCAGAGAGAAUGUCCTG | |
| Sense strand targeting | SEQ ID NO. 14 |
| SEQ ID NO. 6: | |
| 5′-GAACAAACCCGACAGUAAATT | |
| Antisense strand targeting | SEQ ID NO. 15 |
| SEQ ID NO. 6: | |
| 5′-UUUACUGUCGGGUUUGUUCTG | |
| Sense strand targeting | SEQ ID NO. 16 |
| SEQ ID NO. 7: | |
| 5′-CAGGCUUUAUCGUCAUTT | |
| Antisense strand targeting | SEQ ID NO. 17 |
| SEQ ID NO. 7: | |
| 5′-AUGACGAUAAAGCAGCCUGTG | |

Other agents useful in the practice of the present invention are anti-PTPsigma antibodies, or antigen-binding fragments thereof. To produce antibodies against PTPsigma, host animals may be injected with a full-length PTPsigma protein. Hosts may be injected with peptides of different lengths encompassing a desired target sequence. For example, peptide antigens that are at least 5, 10, 15, 20, 25, 30, 35, 40, 45, 50, 55, 60, 65, 70, 75, 80, 85, 90, 95, 100, 105, 110, 115, 120, 125, 130, 135, 140, 145 or 150 amino acids may be used. Alternatively, if a portion of a protein defines an epitope, but is too short to be antigenic, it may be conjugated to a carrier molecule in order to produce antibodies. Some suitable carrier molecules include keyhole limpet hemocyanin, Ig sequences, TrpE, and human or bovine serum albumen. Conjugation may be carried out by methods known in the art. One such method is to combine a cysteine residue of the fragments with a cysteine residue on the carrier molecule.

In addition, antibodies to three-dimensional epitopes, i.e., non-linear epitopes, may also be prepared, based on, e.g., crystallographic data of proteins. Antibodies obtained from that injection may be screened against the short antigens of proteins described herein. Antibodies prepared against a phosphatase peptide may be tested for activity against that peptide as well as the full length phosphatase protein. Antibodies may have affinities of at least about 10⁻⁶M, 10⁻⁷M, 10⁻⁸M, 10⁻⁹M, 10⁻¹⁰M, 10⁻¹¹M or 10⁻¹²M or higher toward the phosphatase peptide and/or the full length phosphatase protein described herein.

Suitable cells for the DNA sequences and host cells for antibody expression and secretion can be obtained from a number of sources, including the American Type Culture Collection “Catalogue of Cell Lines and Hybridomas” 5th edition (1985) Rockville, Md., U.S.A.).

Methods of antibody purification are well known in the art. See, for example, Harlow and Lane (1988) Antibodies: A Laboratory Manual, Cold Spring Harbor Laboratory, N.Y. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-antibody. Antibodies may also be purified on affinity columns according to methods known in the art.

Antibodies to PTPsigma (anti-PTPsigma antibodies) may be prepared as described above to induce autophagy. In a further embodiment, the antibodies to PTPsigma described herein (whole antibodies or antibody fragments) may be conjugated to a biocompatible material, such as polyethylene glycol molecules (PEG) according to methods well known to persons of skill in the art to increase the antibody's half-life. See for example, U.S. Pat. No. 6,468,532. Functionalized PEG polymers are available, for example, from Nektar Therapeutics. Commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the polypeptide, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc.

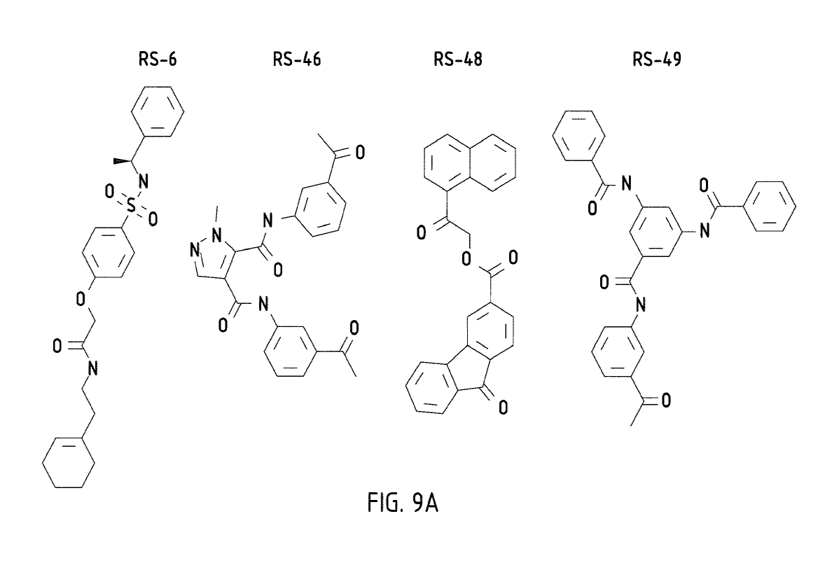

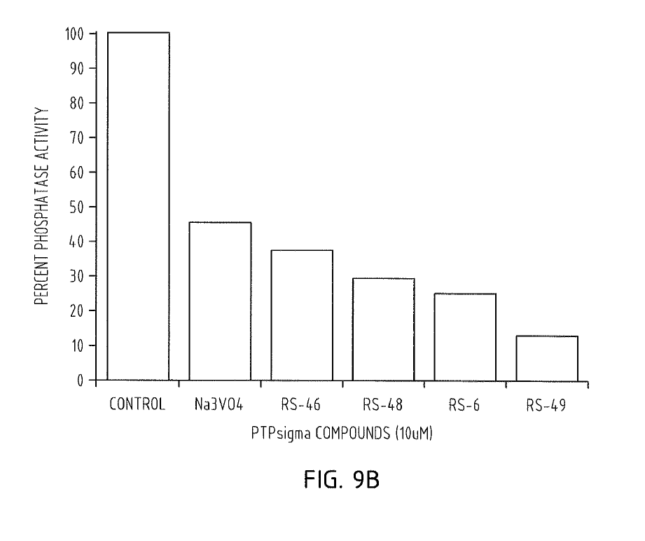

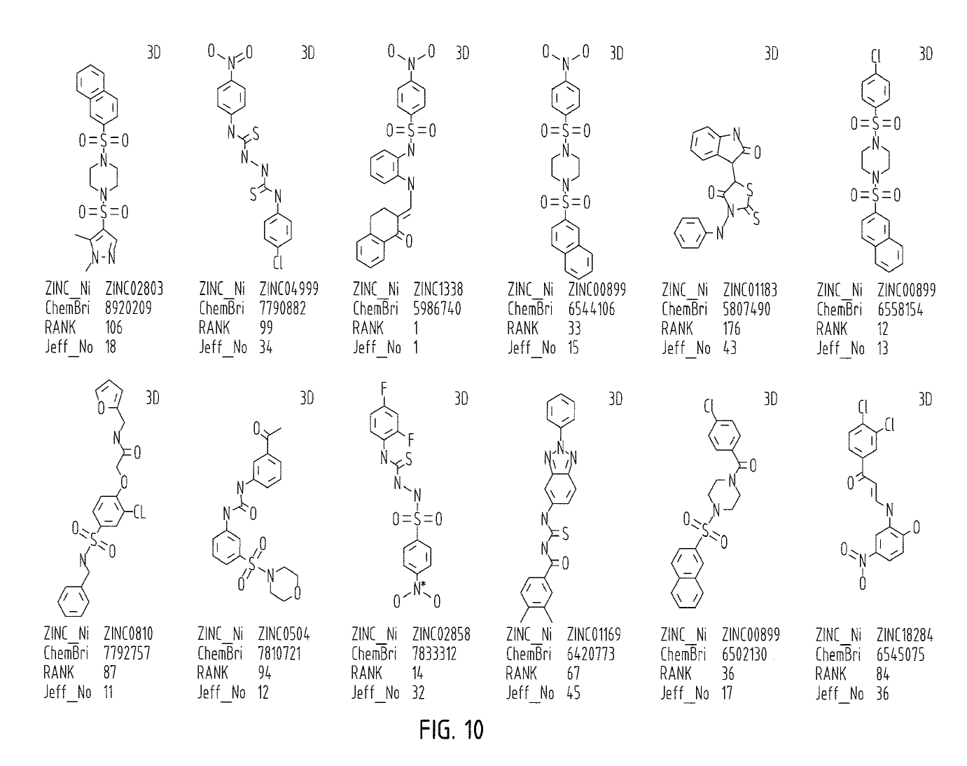

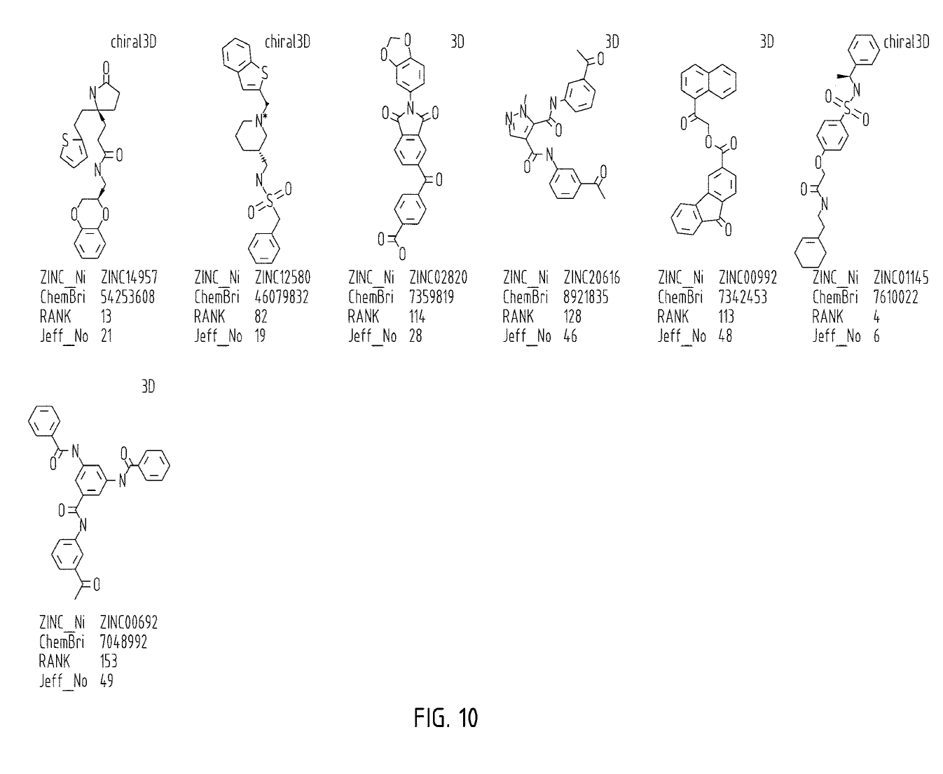

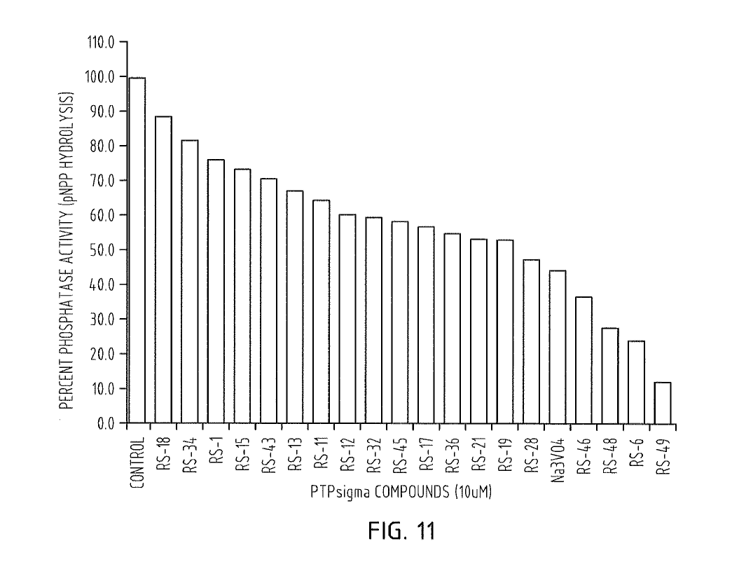

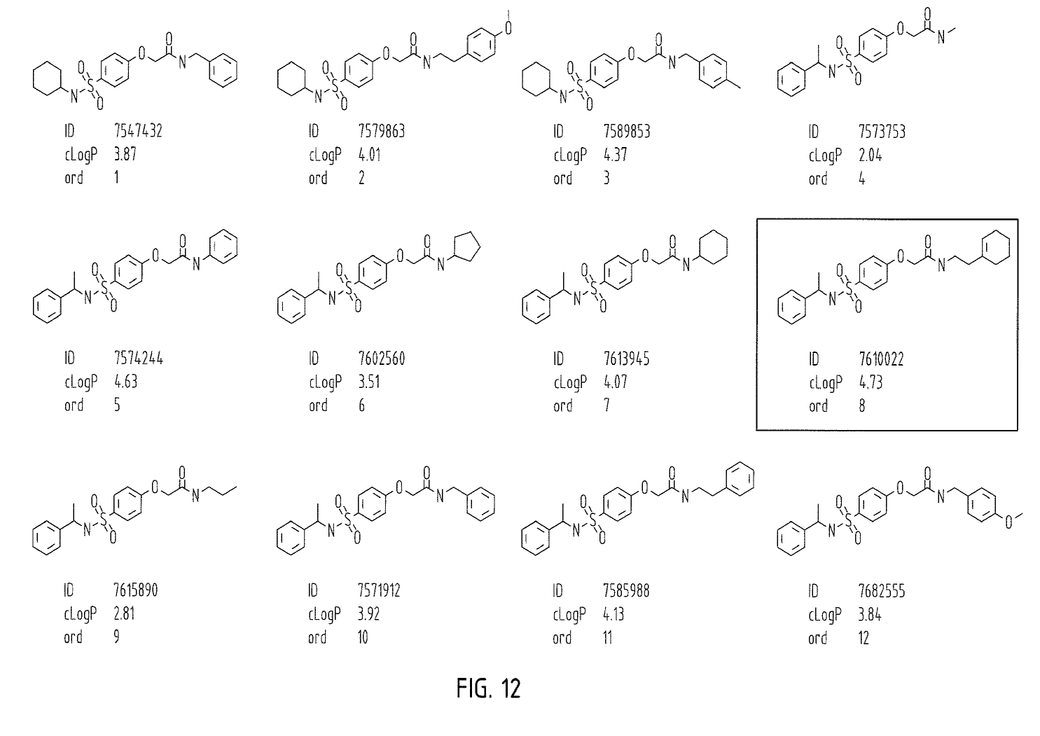

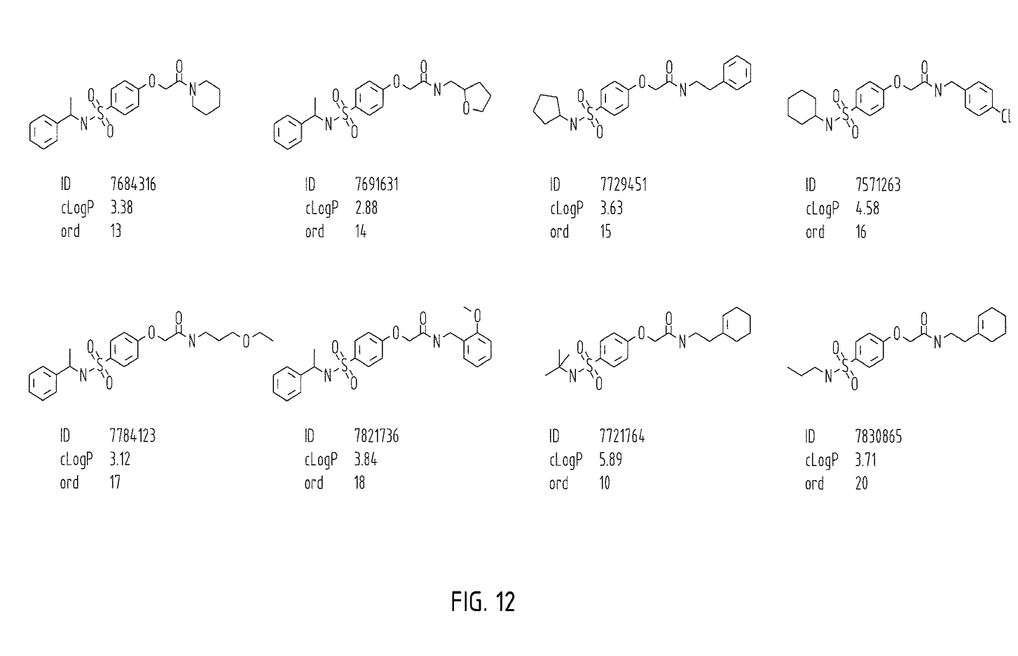

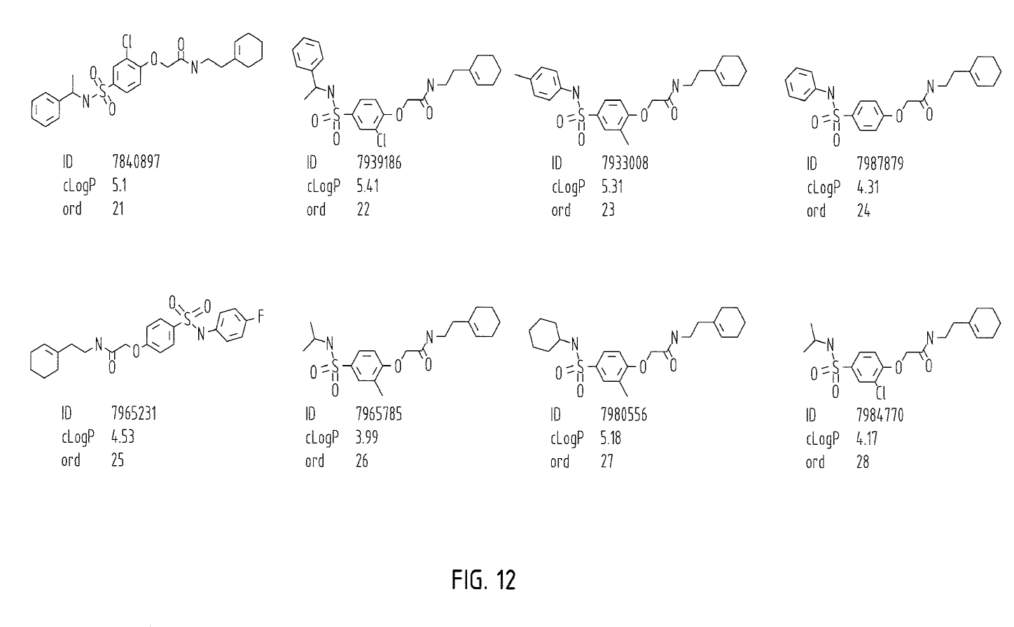

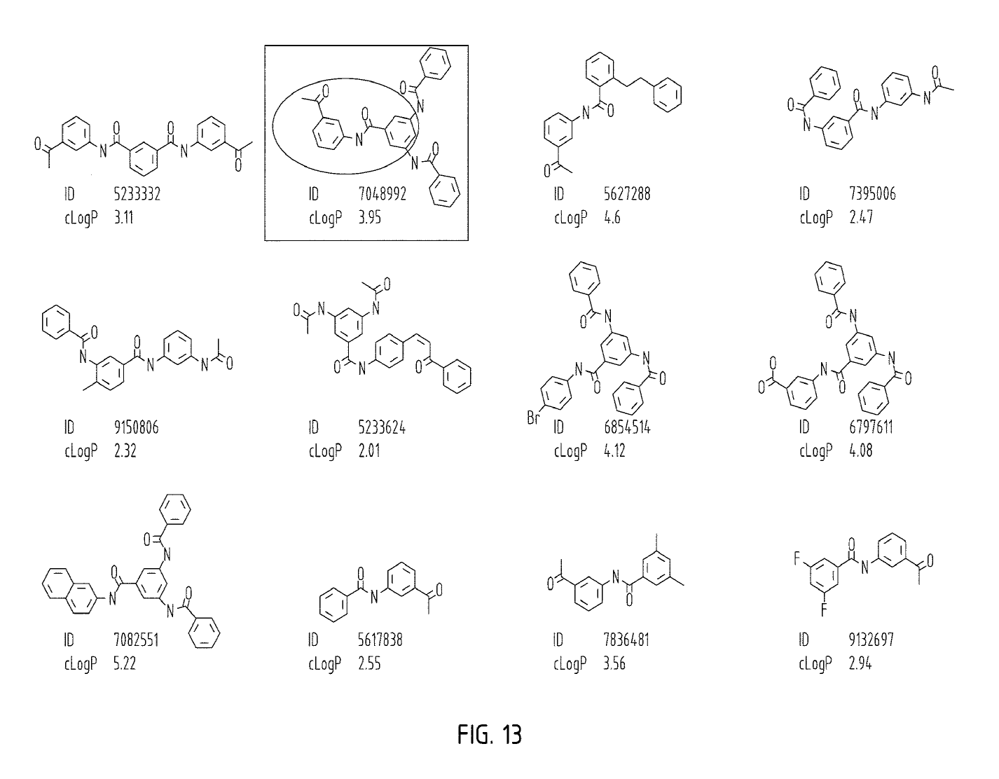

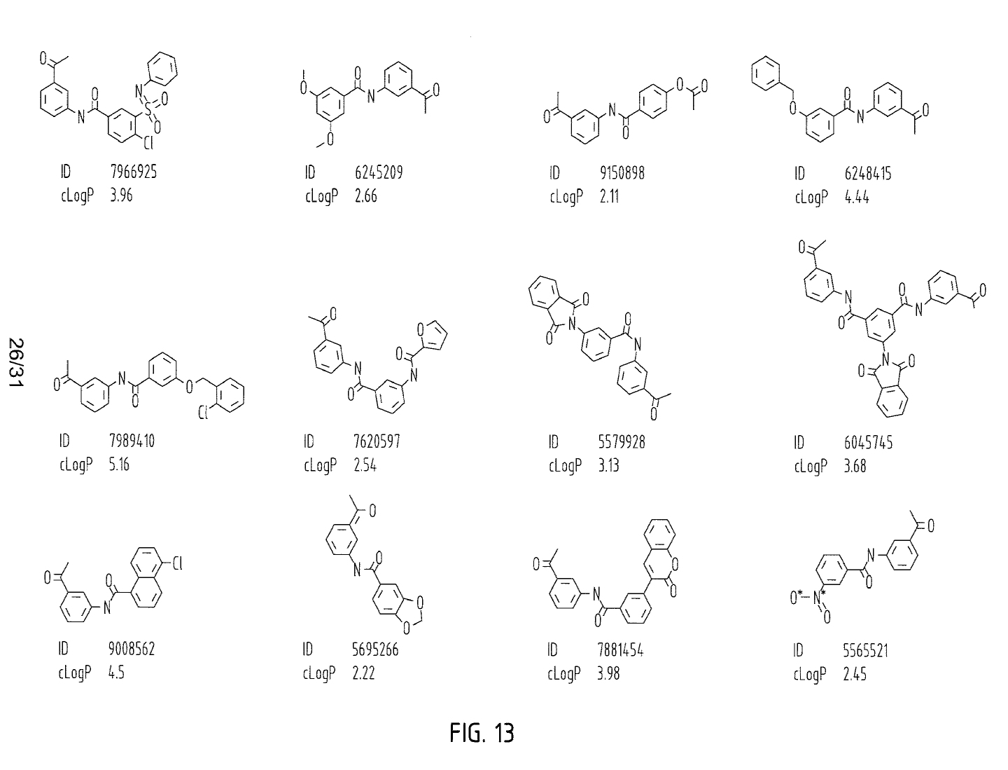

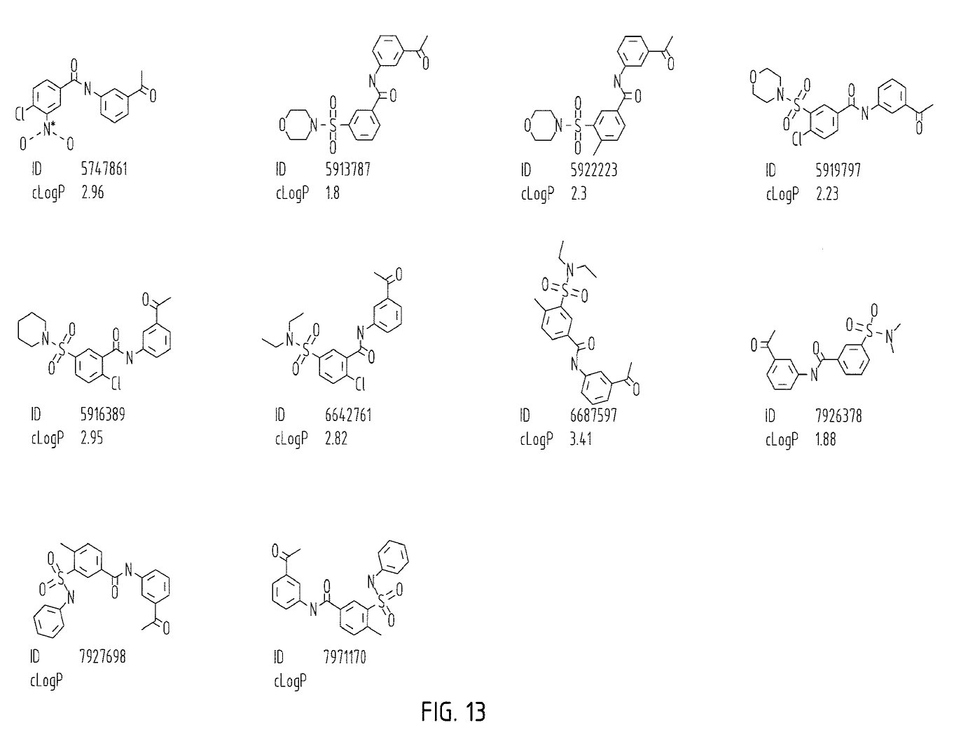

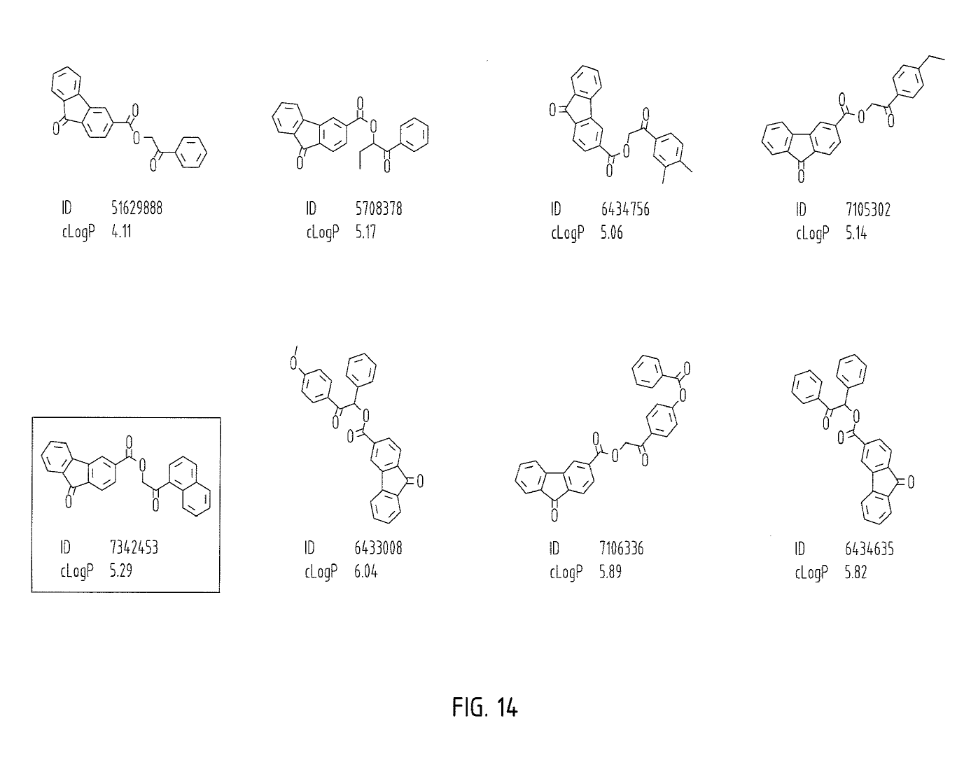

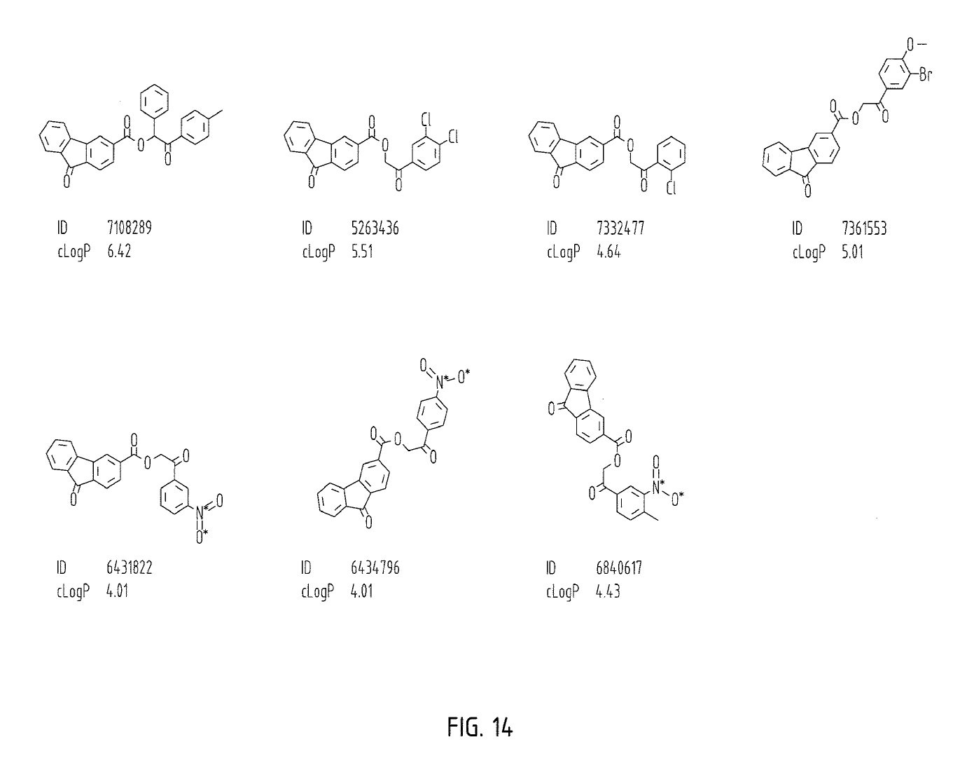

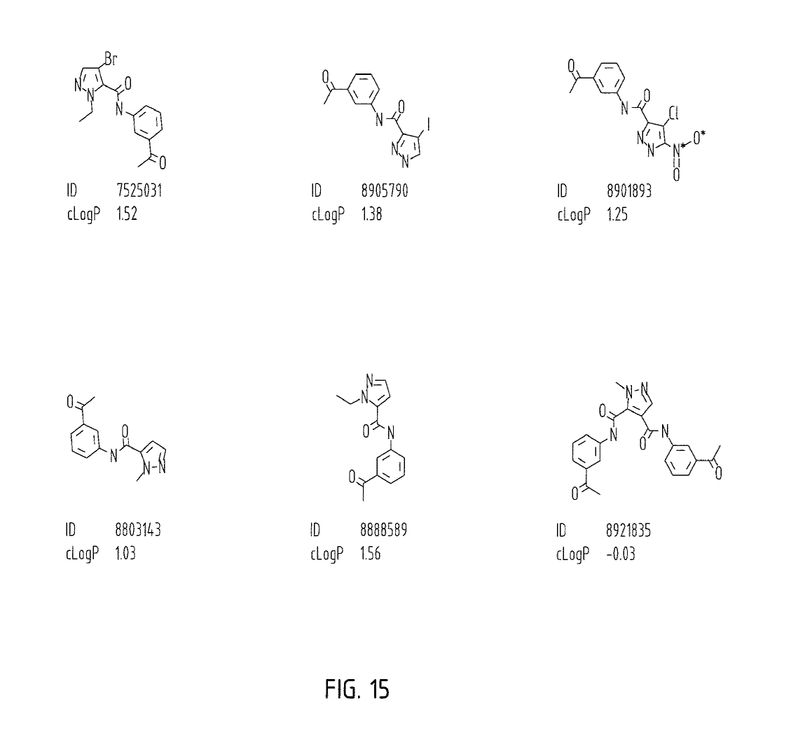

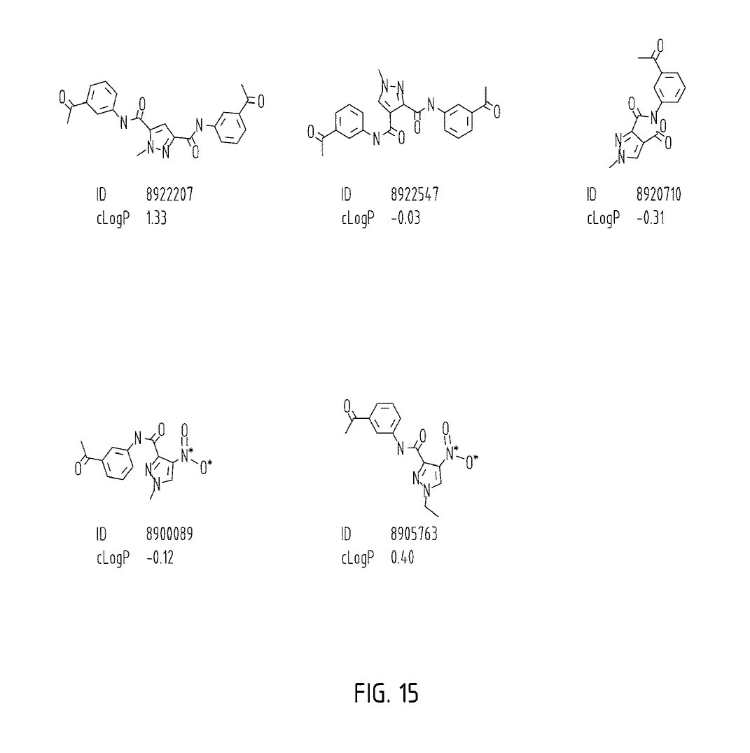

Further, small organic molecules are one type of agent that is useful in practicing the present inventive method; and they also are useful in practicing the methods of modulating autophagy in a cell that are described hereinbelow. Examples of such small organic molecules include nineteen small molecules (FIG. 10). These small molecules exhibited inhibition of PTPRS activity as shown in FIG. 11 (see also, Example 7 below). Note that the designations “RS-” (used in FIGS. 9A, 9B, and 11) and “Jeff_No” (used in FIG. 10) including the same numeral identify the same small molecule, e.g., “RS-6” identifies the same small molecule as “Jeff_No 6”. The inhibition in PTPRS activity of four of the small molecules shown in FIG. 10 (FIG. 9A) also is shown in FIG. 9B. Chemical structures of additional small molecule inhibitors derived from small molecule inhibitors RS-6, RS-49, RS-48, and RS-46 (FIG. 9A), are shown in FIGS. 12-15, respectively. Generally, examples of additional small molecule inhibitors are a sulfonamide, a pyrazole, a ketoester, or a substituted phenyl compound, as follows.

One example of an agent useful in practicing the present inventive methods is a sulfonamide of the formula:

R₁—NH—SO₂—R₂—O—(CH₂)ₙ—CO—NR₃R₄ (I)

where n is 1 thru 3;

where R₁ is:
C₁-C₄ alkyl;
C₃-C₇ cycloalkyl;
phenyl-(CH₂)ₘ— where m is 0 thru 2 and phenyl is optionally substituted with one or two CH₃—, C₂H₅—, F— and Cl—;
phenyl-CH(CH₃)— where phenyl is optionally substituted with CH₃—, C₂H₅—, F— and Cl—;

where R₂ is phenyl optionally substituted with one F—, Cl—, CH₃—, C₂H₅—, and (CH₃)₂CH—;

where R₃ is H—:

where R₄ is:
C₁-C₃ alkyl;
C₃-C₇ cycloalkyl;
—CH₂—CH═CH₂
—(CH₂)_{z}—O—R₅ where z is 1 thru 5 and R₅ is C₁-C₃ alkyl;
—(CH₂)_{w}—R₆ where w is 1 thru 3 and R₆ is tetrahydrofuran or C₃-C₇ cycloalkyl optionally containing one double bond;
—(CH₂)_{w}—R₇ where R₇ is C₁-C₃ alkyl and C₁-C₂ alkoxy and where w is as defined above;

where R₃ and R₄ are taken together with the attached nitrogen atom to form a piperidinyl, piperazinyl, morpholinyl, pyrrolidinyl and pyridinyl ring;

and pharmaceutically acceptable salts thereof.

Another example of an agent useful in practicing the present inventive methods is a pyrazole of the formula:

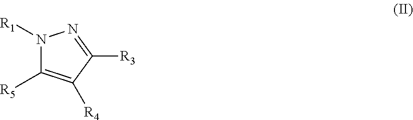

where R₁ is H—, CH₃—, C₂H₅— and cyclo C₃H₅—;

where R₃ is H—, F—, Cl—, Br—, I—, —NO₂, R₃₋₁-phenyl-CO—NH— where R₃₋₁ is CH₃—CO—, CH₃—, C₂H₅—, F—, Cl— and —NO₂;

where R₄ is H—, F—, Cl—, Br—, I—, —NO₂, —CO—O⁻, R₄₋₁-phenyl-CO—NH— where R₄₋₁ is CH₃—CO—, CH₃—, C₂H₅—, F—, Cl— and —NO₂;

where R₅ is H—, F—, Cl—, Br—, I—, —NO₂, R₅₋₁-phenyl-CO—NH— where R₃₋₁ is CH₃—CO—, CH₃—, C₂H₅—, F—, Cl— and —NO₂;

with the proviso:
(1) that one of R₃, R₄ and R₅ must be R₃₋₁-phenyl-CO—NH—, R₄₋₁-phenyl-CO—NH— or R₅₋₁-phenyl-CO—NH—;
   and pharmaceutically acceptable salts thereof.

A further example of an agent useful in practicing the present inventive methods is a ketoester of the formula:

X₁—CO—O—CHR₁—CO—R₂ (III)

where X₁ is fluoren-9-one;

where R₁ is:
H—,
C₁-C₃ alkyl,
phenyl optionally substituted with one or two
   F—,
   Cl,
   —NO₂;

where R₂ is:
1-naphthyl,
2-naphthyl,
phenyl optionally substituted with one or two
   C₁-C₃ alkyl,
   C₁-C₂ alkoxy,
   F—,
   Cl—,
      Br—,
      —NO₂,
      —O—CO-phenyl optionally substituted with 1 F—, Cl— and CH₃—;
         and pharmaceutically acceptable salts thereof.

An additional example of an agent useful in practicing the present inventive methods is a substituted phenyl compound of the formula:

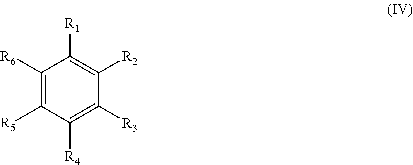

where R₁ is
   —CO—CH₃
   —CO—NH—R₁₋₁ where R₁₋₁ is
      naphthyl
      phenyl optionally substituted with one
         CH₃—CO—
         CH₃—CO—NH—
         phenyl-CO—CH═CH—
         Br—
         Cl—
         ⁻O—CO—;

where R₂ is —H, C₁-C₂ alkyl, —(CH₂)ₘ-phenyl where m is 1 or 2;

and where R₂ and R₃ are taken together with the atoms to which they are attached for form a phenyl ring optionally substituted with one —Cl, —Br and —CH₃;
where R₃ is —H, C₁-C₂ alkyl, —NO₂,
   —CO—NH-phenyl-CO—CH₃,
   —NH—CO—R₃₋₁ where R₃₋₁ is
      phenyl optionally substituted with —O—CO—CH₃,
      C₁-C₃ alkyl,
      2-furanyl,
phthalimide,
coumarin,
—O—CH₂-phenyl optionally substituted with one Cl—, Br— and CH₃—,
—SO₂—NR₃₋₂R₃₋₃ where R₃₋₂ is
   —H,
   C₁-C₃ alkyl and where R₃₋₃ is
   C₁-C₃ alkyl,
   phenyl optionally substituted with one C₁-C₂ alkyl,
   morpholinyl,
   piperidinyl,
   piperazinyl,
and where R₃ and R₄ are taken together with the atoms to which they are attached and —O—CH₂—O— to form a methylene dioxo ring;

where R₄ is H—, Cl—, Br— and C₁-C₂ alkyl;

and where R₄ and R₃ are taken together with the atoms to which they are attached and —O—CH₂—O— to form a methylene dioxo ring;
where R₅ is H—, C₁-C₂ alkyl, —NH—CO-phenyl, —NH—CO-phenyl-CO—CH₃ and —NH—CO—(C₁-C₂ alkyl);
where R₆ is H— and Cl—;
   and pharmaceutically acceptable salts thereof.

Methods of Modulating Autophagy in a Cell

In addition to the above-described treatment methods, the present invention also includes a method of modulating autophagy in a cell. This method comprises administering an agent to a cell such that the expression of PTPRS or PTPsigma is modulated, or such that the biological activity of PTPsigma is modulated; and autophagy in the cell is thereby modulated. In some embodiments, such methods are performed in vitro or ex vivo. The methods, in this regard, may be used to monitor the responsiveness of cells or tissues of a subject (e.g., a human) to such a modulating agent. In some embodiments, the methods are carried out for basic research purposes or clinical research purposes.

In some embodiments, such methods are performed in vivo, such that the cells are in a live animal and the modulating agent is administered to the live animal, e.g., human. When the cell is in a live animal, the method may be a therapeutic method.

According to the principles of the present invention, autophagy in a cell may be modulated either by an antagonist of autophagy or by an agonist of autophagy. An antagonist or inhibitor of autophagy in a cell will lead to a reduction in autophagy in the cell (as compared to a similar cell under similar conditions in the absence of the antagonist or inhibitor); and inhibition of autophagy may lead to at least about a 1.5-fold, 2-fold, 3-fold, 4-fold, 5-fold, or greater fold, increase in autophagy. Conversely, an agonist or activator of autophagy will lead to an increase in autophagy (as compared to a similar cell under similar conditions in the absence of the agonist or activator); and activation of autophagy may lead to at least about a 1.5-fold, 2-fold, 3-fold, 4-fold, 5-fold, or greater fold, increase in autophagy. Whether autophagy in a cell has been modulated can be determined by using the assays known in the art and/or by assays described herein.

Modulating expression of PTPRS or PTPsigma in a cell occurs when the level of expression of PTPsigma is increased or decreased as compared to a control. Suitable controls are described herein and are otherwise known in the art. An increase or decrease in expression of PTPRS or PTPsigma in the cell can be measured by methods known in the art and by those described herein. Modulating the biological activity of PTPsigma occurs when the biological activity of PTPsigma is increased or decreased as compared to a control. Suitable controls are described herein and are otherwise known in the art. An increase or decrease in the biological activity of PTPsigma in the cell can be measured by methods known in the art and by those methods described herein. The biological activity which is modulated may be the phosphatase activity of PTPsigma.

In one embodiment, the agent disrupts the interaction between PTPsigma and phosphatidylinositol 3-phosphate [PI(3)P] or p-Tyr protein. This disruption of this event can be measured by methods known in the art.

A standard yeast two-hybrid assay may be used to assess the effect of a test compound on the PTPsigma-PI(3)P interaction (Mendelsohn and Brent, Curr. Opin. Biotechnol. 5:482-486, 1994). Typically, a vector encoding a synthetic or naturally occurring peptide containing the binding region of the PTPsigma, covalently bound to a DNA binding domain (e.g., GAL4), is transfected into yeast cells containing a reporter gene operably linked to a binding site for the DNA binding domain. Further, a vector encoding either the native partner protein or corresponding binding domain/motif from the binding partner covalently bound to a transcriptional activator (e.g., GaIAD) is also transfected. The effectiveness of a test compound is then assessed by growing the yeast in the presence of the compound and measuring the level of reporter gene expression.

The interaction of the PTPsigma with PI(3)P or p-Tyr protein also may be examined using a GST-fusion protein binding study. A vector encoding a naturally-occurring or synthetic polypeptide containing p-Tyr protein (or PI(3)P lipid) or fragment thereof is fused to GST and expressed in a host cell (e.g., E. coli or Saccharomyces spp.). The GST fusion protein (or lipid) is then contacted with the PTPsigma polypeptide in the presence and absence of a test compound. The PTPsigma may be naturally expressed by the host cell or may be expressed from a second vector inserted into the host cell. Following incubation with the test compound, the host cells are lysed and the GST fusion proteins are recovered using glutathione-Sepharose (GSH-Seph) beads. Typically, the GST fusion proteins are released from the GSH-Seph by boiling and the proteins visualized by electrophoretic separation on an SDS-PAGE gel. A skilled artisan will readily understand that the GST-Pulldown assay described here can be readily adapted to a cell-free assay by incubating the purified GST fusion protein with purified recombinant PTPsigma.

A variety of well known cell-free techniques may be used to assess the effects of a test compound on the interaction between a phosphatase and a partner of interest [e.g., PTPsigma and PI(3)P or p-Tyr protein]. Fluorescence polarization assays are particularly useful for this purpose. In this assay, a peptide (about 6-12 amino acids) containing the binding motif found in the partner(s) has a fluorophore (e.g., fluorescein, BODIPY) conjugated to its N-terminus is incubated in the presence and absence of increasing amounts of recombinant phosphatase (e.g., 0.01-1 μM) for 10 minutes at room temperature. Aliquots from each reaction are placed in a plate black-walled microtiter (e.g., 384-well) plate and polarization measured using an Analyst plate reader. Increasing concentrations of the phosphatase causes an increase in polarization. Titrating in the “free” binding motif peptide (i.e., unconjugated) inhibits the change in polarization, whereas a mutated version of the binding peptide does not. The appearance of low polarization, even in the presence of high concentrations of phosphatase, indicates flexible binding of the binding peptide to the phosphatase and suggests the presence of the propeller effect. Designing shorter dye-conjugated binding peptides usually alleviates this problem. The effect of standard assay variables, including incubation time, temperature, pH (7.2-8.5), and buffers, on polarization is readily controlled during routine assay optimization. This assay is readily adaptable for identifying test compounds that inhibit binding of a phosphatase to partner(s). The use of automated liquid handling systems and plate readers makes this assay readily adaptable to a high-throughput format for screening large numbers of test compounds. For compound screening, the test compound is added to a mixture of the fluorescently labeled binding peptide and the phosphatase. Compounds that inhibit the polarization increase (or cause a decrease in polarization) resulting from increasing amounts of the recombinant phosphatase are therapeutic candidates.

Agents useful in the practice of the present method are capable of modulating the level of PTPRS or PTPsigma, or modulating the biological activity of PTPsigma. Such agents and their manufacture are described herein and include an inhibitory nucleic acid, a small organic molecule, an anti-PTPsigma antibody or antigen-binding fragment thereof, and derivatives thereof. Inhibitory nucleic acids useful in the method of the present invention include siRNAs targeted to any of SEQ ID NOs: 3-7. Further, administering an agent to a cell can be accomplished by techniques known in the art and as described herein.

Screening Assays to Identify Modulators of PTPRS and PTPsigma

The identification of agents or compounds capable of modulating the expression of PTPRS or PTPsigma or the activity of PTPsigma or, alternatively, the identification of proteins and/or signaling molecules that physically bind to PTPsigma or PI(3)P and disrupt PTPsigma-PI(3)P interactions, may be important for treating autophagy disorders and potentiating other treatments. Therefore, it is desirable to identify modulators of PTPRS and PTPsigma for future therapeutic use.

In general, agents or compounds capable of modulating the expression of PTPRS or PTPsigma or the activity of PTPsigma or, alternatively, that disrupt PTPsigma-PI(3)P interactions, may be identified from large libraries of both natural product or synthetic (or semisynthetic) extracts or chemical libraries according to methods known in the art. Those skilled in the field of drag discovery and development will understand that the precise source of agents (e.g. test extracts or compounds) is not critical to the screening procedure(s) of the invention. Accordingly, virtually any number of chemical extracts or compounds can be screened using the methods described herein. Examples of such agents, extracts, or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmnaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to modulate the expression level, phosphatase activity, or binding activity, of PTPRS or PTPsigma further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogeneous extracts are known in the art. If desired, compounds shown to be useful agents for the treatment of pathogenicity are chemically modified according to methods known in the art. Potential modulators of PTPRS and PTPsigma disclosed herein may include organic molecules, nucleic acids, peptides, peptide mimetics, polypeptides, and antibodies that bind to a nucleic acid sequence or polypeptide of the invention and thereby inhibit or extinguish its activity. Potential antagonists also include small organic molecules that bind to and occupy the binding site of the polypeptide thereby preventing binding to cellular binding molecules, such that normal biological activity is prevented. Other potential antagonists include antisense molecules (e.g., siRNAs).

As described herein, a method for identifying a test compound that modulates autophagy in a cell, comprises: (a) providing (i) a PTPsigma polypeptide, or a PTPsigma homolog capable of binding to PI(3)P, and (ii) a test compound for screening; (b) mixing, in any order, the PTPsigma polypeptide, or the homolog, and the test compound; and (c) measuring the biological activity of the PTPsigma polypeptide, or the homolog, in the presence of the test compound as compared to the biological activity of the PTPsigma polypeptide, or the homolog, in the absence of the test compound; wherein a change in the biological activity of the PTPsigma polypeptide, or the homolog, in the presence of the test compound as compared to the absence of the test compound is indicative of a test compound that is an agent capable of modulating autophagy in a cell.

In one aspect, the present method for identifying an agent includes the use of a PTPsigma polypeptide, or a PTPsigma homolog capable of binding to PI(3)P. Methods for making such a polypeptide or homolog are known in the art and/or are described herein.

PTPsigma polypeptides described herein include naturally purified products, products of chemical synthetic procedures, and products produced by recombinant techniques from a prokaryotic or eukaryotic host, including, for example, bacterial, yeast, higher plant, insect, and mammalian cells. The PTPsigma polypeptides may comprise, consist of or consist essentially of an amino acid sequence encoded by a PTPRS nucleotide sequence having accession number NM_{—}002850 (see, SEQ ID NO. 1). The amino acid sequence for PTPsigma is shown in the concurrently filed Sequence Listing as SEQ ID No. 2. Yet other polypeptides comprise, consist of or consist essentially of an amino acid sequence that has at least about 70%, 80%, 90%, 95%, 98% or 99% identity or homology with PTPsigma. For example, polypeptides that differ from a sequence in a naturally-occurring protein in about 1, 2, 3, 4, 5 or more amino acids are also contemplated. The differences may be substitutions, e.g., conservative substitutions, deletions or additions. The differences are preferably in regions that are not significantly conserved among different species. Such regions can be identified by aligning the amino acid sequences from various species. These amino acids can be substituted, e.g., with those found in another species. Other amino acids that may be substituted, inserted or deleted at these or other locations can be identified by mutagenesis studies coupled with biological assays.

Proteins may be used as a substantially pure preparation, e.g., wherein at least about 90% of the protein in the preparation are the desired protein. Compositions comprising at least about 50%, 60%, 70%, or 80% of the desired protein may also be used.

Other proteins that are encompassed herein are those that comprise modified amino acids. Exemplary proteins are derivative proteins that may be one modified by glycosylation, pegylation, phosphorylation or any similar process that retains at least one biological function of the protein from which it was derived.

Proteins may also comprise one or more non-naturally occurring amino acids. For example, nonclassical amino acids or chemical amino acid analogs can be introduced as a substitution or addition into proteins. Non-classical amino acids include, but are not limited to, the D-isomers of the common amino acids, 2,4-diaminobutyric acid, alpha-amino isobutyric acid, 4-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, Calpha-methyl amino acids, Nalpha-methyl amino acids, and amino acid analogs in general. Furthermore, the amino acid can be D (dextrorotary) or L (levorotary). In certain embodiments, a PTPsigma polypeptide may be a fusion protein containing a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

Polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography, lectin chromatography and high performance liquid chromatography (“HPLC”) is employed for purification. Polypeptides of the invention include naturally purified products, products of chemical synthetic procedures, and products produced by recombinant techniques from a prokaryotic or eukaryotic host, including, for example, bacterial, yeast, higher plant, insect and mammalian cells.

In certain embodiments, it may be advantageous to provide naturally-occurring or experimentally-derived homologs of a polypeptide of the invention. Such homologs may function in a limited capacity as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of a polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins.

Polypeptides may be derived from the full-length PTPsigma polypeptide. Isolated peptidyl portions of that polypeptide may be obtained by screening polypeptides recombinantly produced from the corresponding fragment of the nucleic acid encoding such polypeptide. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides of the invention. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO94/02502).

Methods of generating sets of combinatorial mutants of polypeptides of the invention are provided, as well as truncation mutants, and is especially useful for identifying potential variant sequences (e.g. homologs). The purpose of screening such combinatorial libraries is to generate, for example, homologs which may modulate the activity of a polypeptide of the invention, or alternatively, which possess novel activities altogether. Combinatorially-derived homologs may be generated which have a selective potency relative to a naturally-occurring protein. Such homologs may be used in the development of therapeutics.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high throughput analysis as necessary to screen large numbers of degenerate sequences created by combinatorial mutagenesis techniques.

In an illustrative embodiment of a screening assay, candidate combinatorial gene products are displayed on the surface of a cell and the ability of particular cells or viral particles to bind to the combinatorial gene product is detected in a “panning assay”. For instance, the gene library may be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell surface protein, e.g. FITC-substrate, to score for potentially functional homologs. Cells may be visually inspected and separated under a fluorescence microscope, or, when the morphology of the cell permits, separated by a fluorescence-activated cell sorter. This method may be used to identify substrates or other polypeptides that can interact with a PTPsigma polypeptide.

The polypeptides disclosed herein may be reduced to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate.

For instance, derivatives of the phosphatase described herein may be chemically modified peptides and peptidomimetics. Peptidomimetics are compounds based on, or derived from, peptides and proteins. Peptidomimetics can be obtained by structural modification of known peptide sequences using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

With the present method of identifying a test compound, the biological activity of the PTPsigma polypeptide, or the homolog, is measured in the presence of the test compound and compared to the biological activity of the PTPsigma polypeptide, or the homolog, in the absence of the test compound. A change in the biological activity of the PTPsigma polypeptide, or the homolog, in the presence of the test compound as compared to the absence of the test compound is indicative of a test compound that is an agent capable of modulating autophagy in a cell.

Candidate tests compounds may be an inhibitory nucleic acid, a small organic molecule, an anti-PTP sigma antibody or antigen-binding fragment thereof, and derivatives thereof.

Further, assays to evaluate the biological activity of an enzyme, such as a phosphatase are well known by those of skill in the art and/or are described herein. The biological activity assayed may be the phosphatase activity of PTPsigma or the homolog. One such assay is the ProFluor™ Tyrosine Phosphatase Assay (Promega Corporation). This assay may be used to measure the biological activity of a tyrosine phosphatase, such as PTPsigma, using a purified enzyme. The assay may be initiated with a standard phosphatase reaction performed in the provided reaction buffer that contains a bisamide rhodamine 110 phosphopeptide substrate (PTPase RIIO Substrate) and a Control AMC Substrate that serves as a control for compounds that may inhibit the protease. In this configuration, both the PTPase RI IO Substrate and Control AMC Substrate are nonfluorescent. Following the phosphatase reaction, addition of a protease solution simultaneously stops the phosphatase reaction and completely digests the nonphosphorylated PTPase RI IO Substrate and the Control AMC substrate, producing highly fluorescent rhodamine 110 and AMC. The phosphorylated substrate, however, is resistant to digestion by the Protease Reagent and remains nonfluorescent. Thus, the measured fluorescence intensity in the assay correlates with phosphatase activity. The fluorescent signal is very stable (<20% change of fluorescence intensity over 4 hours), allowing batch-plate reading. The assay produces Z′-factor values greater than 0.7 in either 96-well (data not shown) or 384-well plate formats, and it identifies known phosphatase inhibitors and may be used to identify inhibitors in a screen of library compounds. The assay produces IC50 values for known inhibitors that are comparable to those reported in literature.

The activity of a phosphatase protein, fragment, or variant thereof may be assayed using an appropriate substrate or binding partner or other reagent suitable to test for the suspected activity. For catalytic activity, the assay is typically designed so that the enzymatic reaction produces a detectable signal. For example, mixture of a kinase with a substrate in the presence of ³²P will result in incorporation of the ³²P into the substrate. The labeled substrate may then be separated from the free ³²P and the presence and/or amount of radiolabeled substrate may be detected using a scintillation counter or a phosphorimager. Similar assays may be designed to identify and/or assay the activity of a wide variety of enzymatic activities. Based on the teachings herein, the skilled artisan would readily be able to develop an appropriate assay for a polypeptide of the invention.

In another embodiment, the activity of a polypeptide may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes. Alternatively, it may be desirable to measure the overall rate of DNA replication, transcription and/or translation in a cell. In general this may be accomplished by growing the cell in the presence of a detectable metabolite which is incorporated into the resultant DNA, RNA, or protein product. For example, the rate of DNA synthesis may be determined by growing cells in the presence of BrdU which is incorporated into the newly synthesized DNA. The amount of BrdU may then be determined histochemically using an anti-BrdU antibody.

Additionally, the present invention includes a second screening method, i.e., a method for identifying a test compound that modulates autophagy comprising (a) providing (i) a cell comprising a nucleic acid, or a fragment thereof, that encodes PTPsigma, or a PTPsigma homolog capable of binding to PI(3)P or p-Tyr protein, and (ii) a test compound; (b) contacting the test compound and the cell; and (c) measuring the expression of the PTPsigma protein, or the homolog, in the cell in the presence of the test compound as compared to the expression of the PTPsigma protein, or homolog, in the cell in the absence of the test compound; wherein a change in expression of the PTPsigma protein, or homolog, in the cell in the presence of the test compound is indicative of a test compound that modulates autophagy. In further embodiments, the method may include an additional step of testing for autophagy; and the test compound may increase or decrease autophagy in the cell.

A cell including a nucleic acid, or a fragment thereof, that encodes PTPsigma, or a PTPsigma homolog capable of binding to PI(3)P, is used in the present method. Methods for making such a cell are known in the art and/or are described herein.

One aspect of the present invention includes a cell comprising a nucleic acid, or fragment thereof, that encodes PTPsigma, or a PTPsigma homolog capable of binding to PI(3)P or p-Tyr protein. In one embodiment, this nucleic acid is used in a method for identifying a test compound that modulates autophagy. Accordingly, described herein is a nucleic acid that encodes human PTPsigma. The cDNA sequence for PTPRS (GenBank Accession No. NM_{—}002850) is shown in the concurrently filed Sequence Listing as SEQ ID NO. 1. Nucleic acids used in methods of the present invention may also comprise, consist of or consist essentially of any of the nucleotide sequences described herein. Yet other nucleic acids comprise, consist of or consist essentially of a nucleotide sequence that has at least about 70%, 80%, 90%, 95%, 98% or 99% identity or homology with the PTPRS gene described herein. Substantially homologous sequences may be identified using stringent hybridization conditions.

Isolated nucleic acids which differ from the nucleic acids used with methods of the invention due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the polypeptides of the invention will exist. One skilled in the art will appreciate that these variations in one or more nucleotides (from less than 1% up to about 3 or 5% or possibly more of the nucleotides) of the nucleic acids encoding a particular protein of the invention may exist among a given species due to natural allelic variation. Any and all such nucleotide variations and resulting amino acid polymorphisms are within the scope of nucleic acids used with the methods of this invention. Bias in codon choice within genes in a single species appears related to the level of expression of the protein encoded by that gene. Accordingly, the invention encompasses nucleic acid sequences which have been optimized for improved expression in a host cell by altering the frequency of codon usage in the nucleic acid sequence to approach the frequency of preferred codon usage of the host cell. Due to codon degeneracy, it is possible to optimize the nucleotide sequence without affecting the amino acid sequence of an encoded polypeptide. Accordingly, any nucleotide sequence that encodes all or a substantial portion of the amino acid sequence of polypeptides of the invention is within the scope of the invention.

Nucleic acids encoding proteins which have amino acid sequences evolutionarily related to a polypeptide disclosed herein are provided, wherein “evolutionarily related to”, refers to proteins having different amino acid sequences which have arisen naturally (e.g. by allelic variance or by differential splicing), as well as mutational variants of the proteins of the invention which are derived, for example, by combinatorial mutagenesis.

Fragments of nucleic acids encoding PTPsigma, or a PTPsigma homolog capable of binding to PI(3)P or p-Tyr protein, are also provided. As used herein, a fragment of a nucleic acid encoding an active portion of a polypeptide disclosed herein refers to a nucleotide sequence having fewer nucleotides than the nucleotide sequence encoding the full length amino acid sequence of a polypeptide of the invention, and which encodes a given polypeptide that retains at least a portion of a biological activity of the full-length PTPsigma protein as defined herein, or alternatively, which is functional as a modulator of the biological activity of the full-length protein. For example, such fragments include a polypeptide containing a domain of the full-length protein from which the polypeptide is derived that mediates the interaction of the protein with another molecule (e.g., polypeptide, DNA, RNA, etc.).

Nucleic acids provided herein may also contain linker sequences, modified restriction endonuclease sites and other sequences useful for molecular cloning, expression or purification of such recombinant polypeptides.

A nucleic acid encoding a PTPsigma may be obtained from mRNA or genomic DNA from any organism in accordance with protocols described herein, as well as those generally known to those skilled in the art. A cDNA encoding a polypeptide of the invention, for example, may be obtained by isolating total mRNA from an organism, for example, a bacteria, virus, mammal, etc. Double stranded cDNAs may then be prepared from the total mRNA, and subsequently inserted into a suitable plasmid or bacteriophage vector using any one of a number of known techniques.

A gene encoding PTPsigma may also be cloned using established polymerase chain reaction techniques in accordance with the nucleotide sequence information provided by the invention. In one aspect, methods for amplification of a nucleic acid of the invention, or a fragment thereof may comprise: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid.

Host cells may be transfected with a recombinant gene in order to express a desired phosphatase polypeptide. The host cell may be any prokaryotic or eukaryotic cell. For example, a polypeptide may be expressed in bacterial cells, such as E. coli, insect cells (baculovirus), yeast, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the polypeptide will be known to those in the art.

Thus, a nucleotide sequence encoding all or a selected portion of the PTPsigma polypeptide may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology.

Expression vehicles for production of a recombinant protein include plasmids and other vectors. For instance, suitable vectors for the expression of a polypeptide of the invention include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, ρYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-I), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, IU.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs. When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J Bacteriol. 169:751-757) and Salmonella typhimurium and its in vitro activity has been demonstrated on recombinant proteins (Miller et al., (1987) PNAS USA 54:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al).

Coding sequences for a PTPsigma polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

The present method includes measuring the expression of the PTPsigma protein, or the homolog, in the cell in the presence of the test compound as compared to the expression of the PTPsigma protein, or homolog, in the cell in the absence of the test compound; wherein a change in expression of the PTPsigma protein, or homolog, in the cell in the presence of the test compound is indicative of a test compound that modulates autophagy. In further embodiments, the method may include an additional step of testing for autophagy; and the test compound may increase or decrease autophagy in the cell. Methods for measuring autophagy are described herein and otherwise known in the art.

In certain embodiments, test compounds useful in the present invention may be tested for their affect on the expression of the PTPRS nucleic acid or the PTPsigma polypeptide. In an exemplary assay, cells expressing PTPsigma may be treated with a compound(s) of interest, and then assayed for the effect of the compound(s) on PTPRS nucleic acid or PTPsigma protein expression. For example, total RNA may be isolated from cells cultured in the presence or absence of a test compound, using any suitable technique such as the single-step guanidinium-thiocyanate-phenol-chloroform method described in Chomczynski et al. (1987) Anal. Biochem. 162:156-159. The expression of PTPsigma may then be assayed by any appropriate method such as Northern blot analysis, polymerase chain reaction (PCR), reverse transcription in combination with polymerase chain reaction (RT-PCR), and reverse transcription in combination with ligase chain reaction (RT-LCR). Northern blot analysis may be performed as described in Harada et al. (1990) Cell 63:303-312. Briefly, total RNA is prepared from cells cultured in the presence of a test compound. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. A DNA sequence encoding PTPRS may be labeled according to any appropriate method (such as the ³²P-multiprimed DNA labeling system (Amersham)) and used as probe. After hybridization overnight, the filter is washed and exposed to x-ray film. Moreover, a control can also be performed to provide a baseline for comparison. In the control, the expression of PTPsigma may be quantitated in the absence of the test compound.

Alternatively, the levels of mRNA encoding PTPsigma polypeptides may also be assayed, for example, using the RT-PCR method described in Makino et al. (1990) Technique 2:295-301. Briefly, this method involves adding total RNA isolated from cells cultured in the presence of a test agent, in a reaction mixture containing a RT primer and appropriate buffer. After incubating for primer annealing, the mixture may be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification may be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands may be quantified using an imaging analyzer. RT and PCR reaction ingredients and conditions, reagent and gel concentrations, and labeling methods are well known in the art. Variations on the RT-PCR method will be apparent to the skilled artisan. Other PCR methods that can detect the PTPRS nucleic acid can be found in PCR Primer: A Laboratory Manual (Dieffenbach et al. eds., Cold Spring Harbor Lab Press, 1995). A control can also be performed to provide a baseline for comparison. In the control, the expression of mRNA encoding PTPsigma polypeptides may be quantitated in the absence of the test compound.

Alternatively, the expression of PTPsigma polypeptides described herein may be quantitated following the treatment of cells with a test compound using antibody-based methods such as immunoassays. Any suitable immunoassay can be used, including, without limitation, competitive and non-competitive assay systems using techniques such as western blots, radioimmunoassays, ELISA (enzyme-linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, PTPsigma polypeptides described herein may be detected in a sample obtained from cells treated with a test compound, by means of a two-step sandwich assay. In the first step, a capture reagent (e.g., an antibody directed to PTPsigma) is used to capture the specific polypeptide. The capture reagent can optionally be immobilized on a solid phase. In the second step, a directly or indirectly labeled detection reagent is used to detect the captured marker. In one embodiment, the detection reagent is an antibody. The amount of a PTPsigma present in cells treated with a test agent can be calculated by reference to the amount present in untreated cells.

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H).

Examples of suitable fluorescent labels include a fluorescein label, an isothiocyanate label, a rhodamine label, a phycoerythrin label, a phycocyanin label, an allophycocyanin label, an o-phthaldehyde label, and a fluorescamine label.

Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase. Examples of chemiluminescent labels include a luminol label, an isoluminol label, an aromatic acridinium ester label, an imidazole label, an acridinium salt label, an oxalate ester label, a luciferin label, a luciferase label, and an aequorin label.

As will be appreciated by those in the art, the type of host cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. Cell types implicated in a wide variety of disease conditions are particularly useful. Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include those described in the Examples herein, and known research cells, including, but not limited to, HeLa cells, Jurkat T cells, NIH3T3 cells, CHO, Cos, etc. See the ATCC cell line catalog, hereby expressly incorporated by reference.

### Diagnostic Markers and Assays

Under-expression, over-expression, and/or mutation of PTPsigma may be used as a biomarker for diagnosis of an autophagy-related disorder, such a neurodegenerative disorder, an auto-immune disorder, a cardiovascular disorder, a metabolic disorder, hamartoma syndrome, a genetic muscle disorder, a myopathy, and/or a cancer.

Neuronal loss, which is a hallmark of neurodegenerative diseases, is mediated by defective autophagic pathways. Autophagy also occurs in acute pathologies, including ischemia, stroke, spinal cord injuries. Further, decreased levels of autophagy are observed in various neuropathologies, including Parkinson's disease, Alzheimer's disease, amyothrophic lateral sclerosis (ALS), denervation atrophy, otosclerosis, stroke, dementia, multiple sclerosis, Huntington's disease and encephalopathy associated with acquired immunodeficiency disease (AIDS). Since nerve cells generally do not divide in adults and, therefore, new cells are not available to replace the dying cells, the nerve cell death occurring in such diseases results in the progressively deteriorating condition of subjects suffering from the condition. Overexpression and/or mutation of phosphatases as well as the underexpression and/or mutation of phosphatases may serve as markers of acute and/or chronic neuropathologies.

Similarly, autophagy is a critical step in the pathogenesis of several cardiovascular diseases, including, but not limited to myocardial infarction, heart failure, and atherosclerosis as well as other diseases including muscular dystrophy, inflammatory bowel disease, Crohn's disease, autoimmune hepatitis, hemochromatosis, Wilson disease, viral hepatitis, alcoholic hepatitis, glomerulosclerosis, and Monckeberg's medical syndrome. Thus, overexpression and/or mutation of phosphatases as well as the under-expression and/or mutation of phosphatases may serve as markers of cardiovascular diseases as well as other diseases.

Further, the under- or over-expression and/or mutation of a phosphatase may be used to identify subject populations for clinical trials related to cancer, neurodegenerative disease, and/or cardiovascular disease. As such, this information may be used to enable clinicians to determine the most appropriate therapies for each subject, thus improving subject quality of life and increasing and survival.

Expression of a marker for cancer, neurodegenerative disease, and/or cardiovascular may be determined from a biological sample from a subject using a variety of assays known in the art. Exemplary assays to monitor expression of a marker may include, but are not limited to, immunoassays, Northern blot, and in situ hybridization. Biological samples that may be obtained from a subject include, but are not limited to, tissue (e.g., healthy, diseased, and/or tumor tissue), whole blood, plasma, urine, interstitial fluid, lymph, gastric juices, bile, serum, saliva, sweat, and spinal and brain fluids. Furthermore, a biological sample may be either processed (e.g., serum) or present in its natural form.

Tumors that may be diagnosed with the present invention include, but are not limited to, tumors of the breast, colon, lung, liver, lymph node, kidney, pancreas, prostate, ovary, endometrium, spleen, small intestine, stomach, skin, testes, head and neck, esophagus, brain (glioblastomas, medulloblastoma, astrocytoma, oligodendroglioma, ependymomas), blood cells, bone marrow, blood cells, blood or other tissue. The tumor may be distinguished as metastatic or non-metastatic. The methods and combinations of the present invention may also be used for the diagnosis of neoplasia disorders selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondrosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastema, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

Thus, the present invention includes a method of determining whether a subject is suffering from or is at risk for an autophagy-related disorder, including: (a) providing a biological sample obtained from a subject; and (b) determining whether the level of expression of PTPRS nucleic acid or PTPsigma polypeptide in the biological sample differs from the PTPRS or PTPsigma level of expression in a comparable biological sample obtained from a healthy subject.

Pharmaceutical Compositions

An additional aspect of the invention relates to pharmaceutical compositions, including a pharmaceutically acceptable carrier, for any of the therapeutic effects discussed above. Such pharmaceutical compositions comprise an effective amount of an agent capable of modulating the expression of PTPRS or PTPsigma, or modulating the biological activity of PTPsigma, and a pharmaceutically acceptable carrier.

The pharmaceutical compositions may for comprise antibodies, mimetics, agonists, antagonists, or inhibitory nucleic acids in accordance with the present invention. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a subject alone, or in combination with other agents, drugs or hormones.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the subject.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, fragments thereof, antibodies, agonists, antagonists or inhibitors which ameliorates the symptoms or conditions of disorders relating to aberrant autophagy. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the subject, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Having now generally described the invention, the same will be more readily understood through reference to the following examples, which are provided by way of illustration, and are not intended to be limiting of the present invention, unless specified.

### EXAMPLES

### Example 1

### Materials and Methods for Examples 2-6

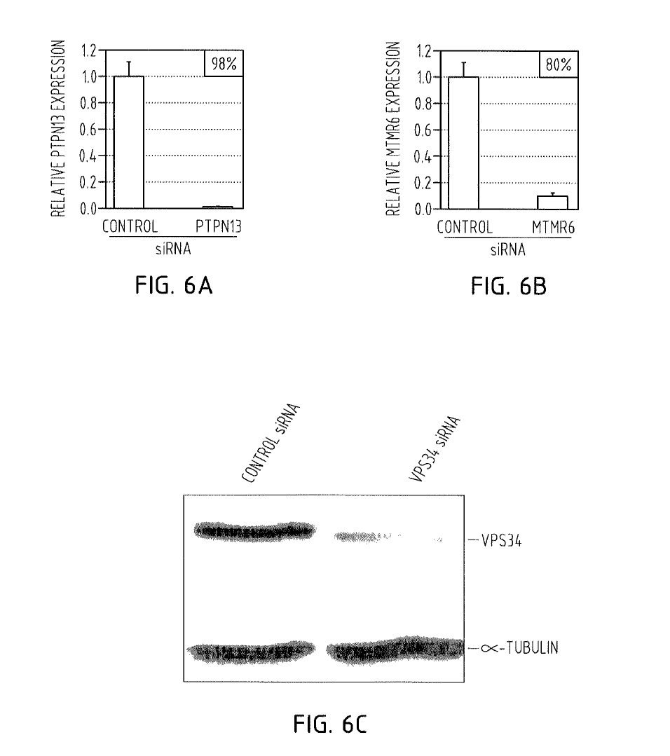

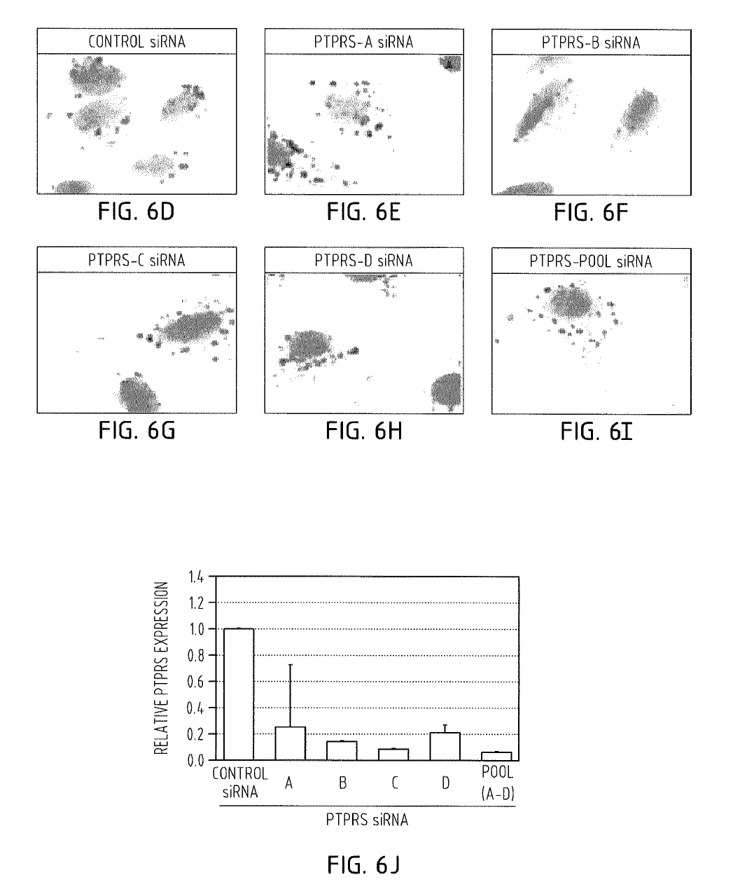

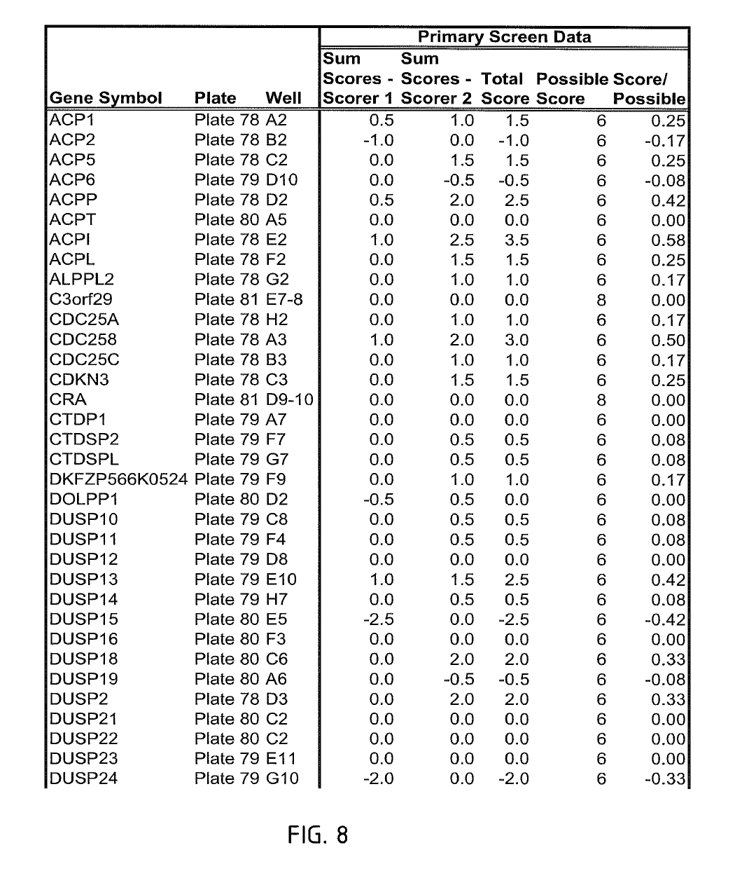

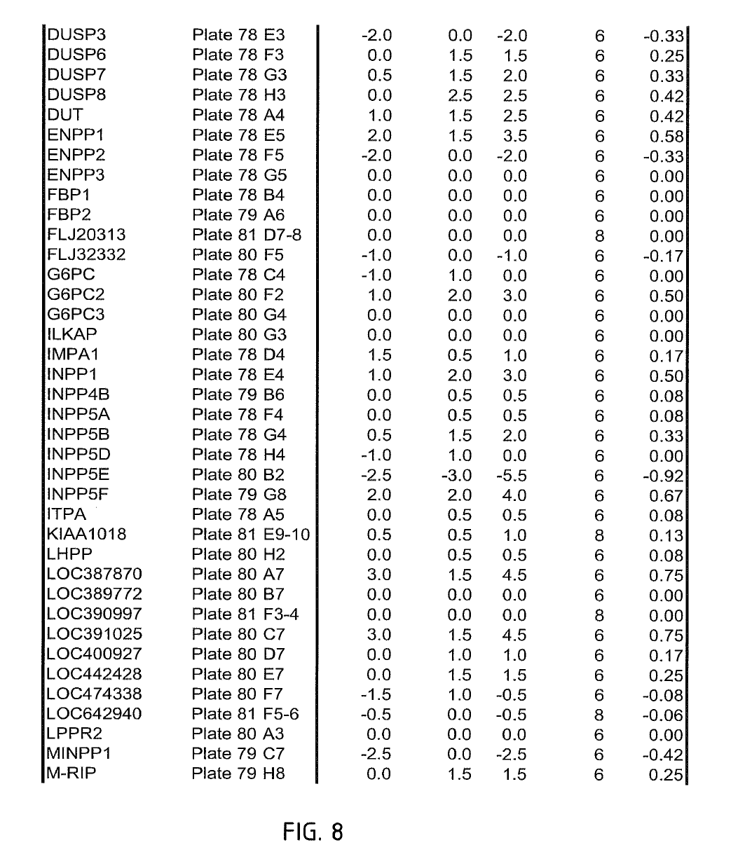

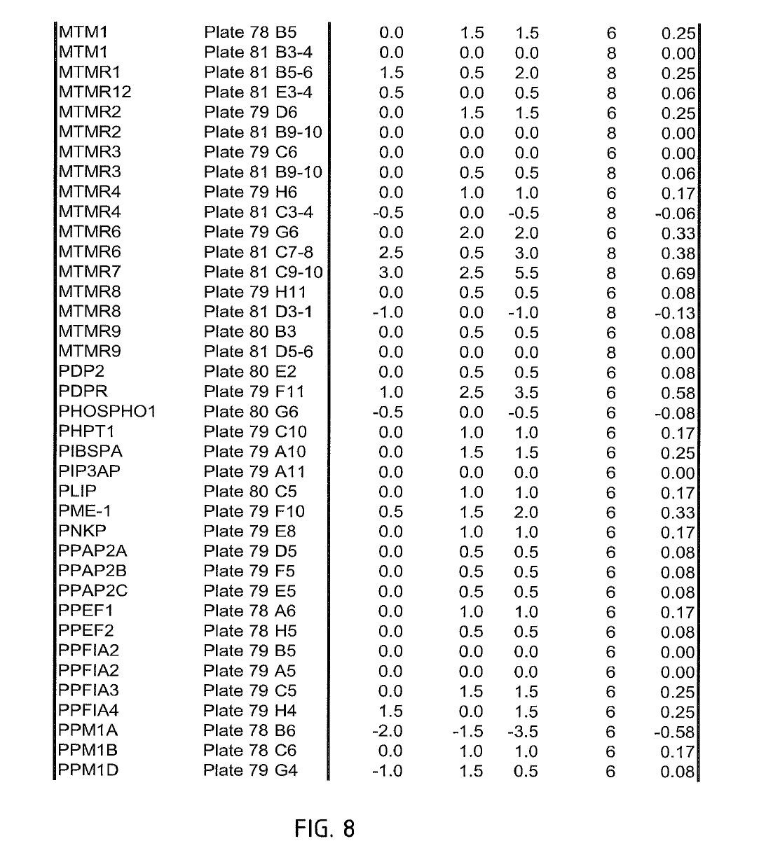

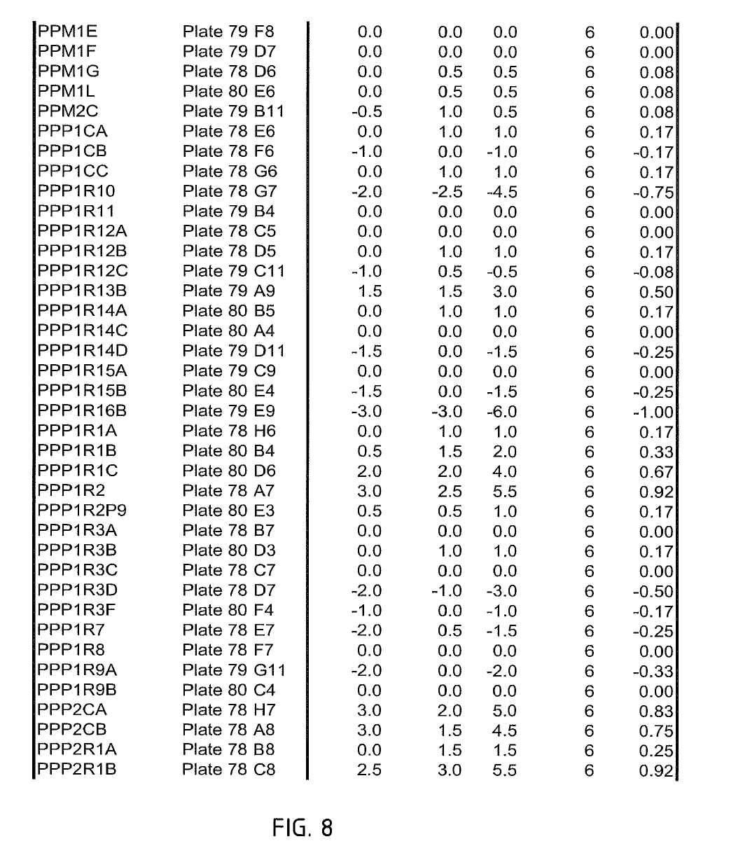

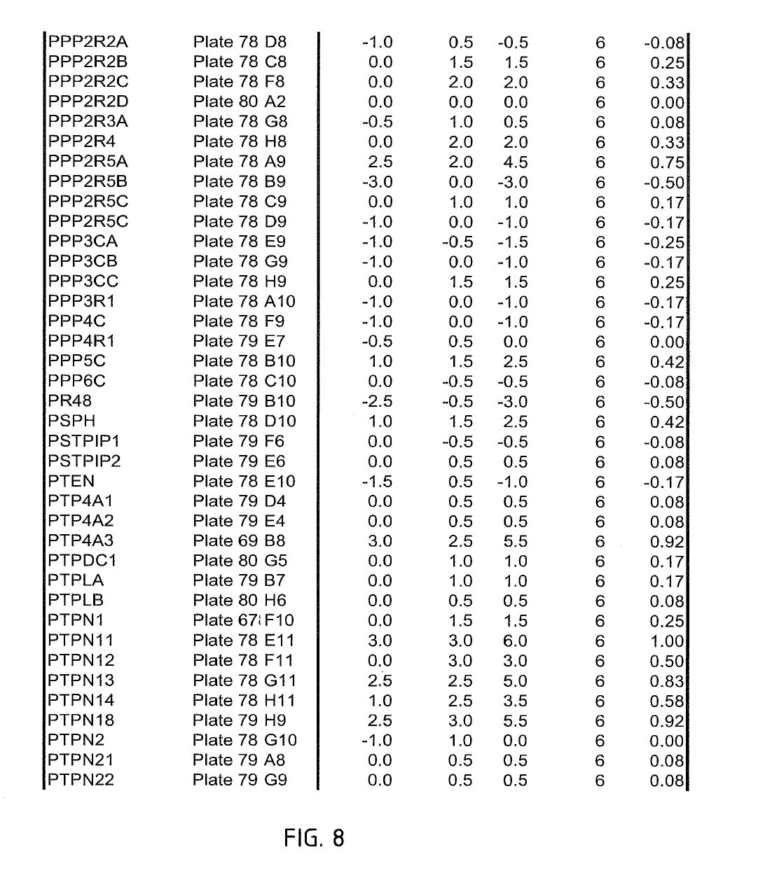

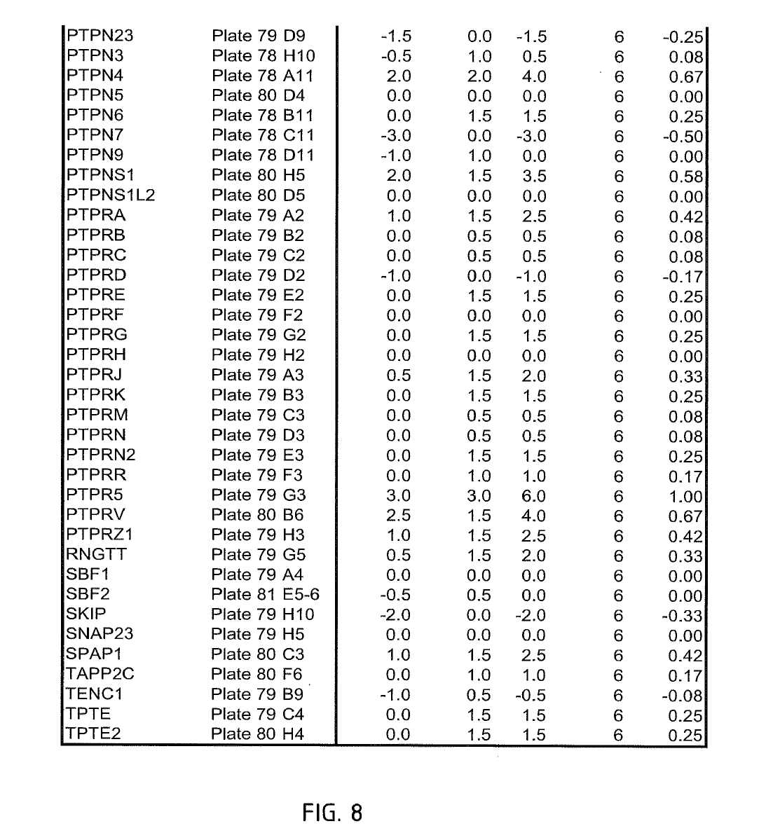

siRNA screen and validation. U2OS-2xFYVE-EGFP cells were seeded on 96-well plates (2,000 per well) in McCoy's medium with 10% fetal bovine serum (FBS) for 24 h. Four siRNAs per phosphatase gene (Qiagen phosphatase library v2.0) were transfected per well at a final concentration of 25 nM using 0.2 μl HiPerfect transfection reagent (Qiagen) per well. At 48 h, cells were fixed with 3.7% formaldehyde and nuclei were stained with Hoechst-33342 (Invitrogen). Cells were visualized at 40× on a Zeiss LSM 510 Meta confocal microscope and 2xFYVE-EGFP distribution was compared to that of control siRNA-transfected cells within each plate. Triplicate wells from each gene were independently scored on a scale from −1 (decreased 2xFYVE-EGFP granularity) to +1 (increased granularity) and mean scores were determined (FIG. 8). Twenty-seven phosphatase genes whose knockdown increased granularity in the primary screen were used in a secondary screen, where four siRNAs were individually transfected to eliminate off-target hits. Quantitative real-time PCR (qRT-PCR) assays with gene-specific primers confirmed that siRNAs effectively reduced mRNA expression of target genes (FIG. 6).

Stuctural modeling analyses. The crystal structures of PTPsigma (PDB 2fh7), MTMR2 (PDB 1zsq), and PTP1B (PDB 1sug) were retrieved from the Protein Data Bank (PDB). The initial conformations of PI(3)P and p-Tyr peptide were extracted from the MTMR2-PI(3)P complex structure (PDB code: 1zsq) and the CD45 pTyr peptide complex structure (PDB 1ygu). The ICM program was used for protein and ligand preparation. PI(3)P and p-Tyr peptide were docked into the active site of PTPsigma and PTP1B with default parameters implemented in the ICM program.

Phospholipid labeling, extraction, and thin layer chromatography (TLC). U2OS cells were seeded at 200,000 cells per well of 6-well tissue culture plates and were transfected with control or PTPRS siRNAs for 48 h. The medium was replaced with phosphate-free DMEM supplemented with 10% phosphate-free FBS for 30 min. ³²PO4 (0.25 mCi) was added per ml of medium for an additional 2 h. Radiolabeling was quenched with ice-cold TCA (10% final concentration) and cells were incubated on ice for 1 h. Cells were scraped, pelleted, and lipids extracted via an acidified Bligh and Dyer method (REF). Lipids were lyophilized, resuspended in chloroform:methanol (1:1), spotted on silica gel TLC plates, and resolved in a chamber using boric acid buffer (REF, P. Majerus). The TLC plate was exposed to film for 20 h at −80° C.

In vitro phosphatase assays. Recombinant proteins (PTPsigma, BC104812 aa1156-1501; MTMR6, NM_{—}004685.2) tagged N-terminally to glutathione-S-transferase (GST) were expressed and purified. PTP1B was from Upstate. Purified proteins were incubated in reaction buffer of 50 mM Tris-HCl, 25 mM sodium acetate, and 10 mM DTT at pH 6. PTPsigma reactions included 0.5 mM MnCl2. Reactions began with the addition of 200 μM water-soluble diC8-PI(3)P (Echelon) or 100 μM phosphotyrosine peptide (Upstate) and incubation was at 37° C. for 30 min. Reactions were quenched by adding 100 μl malachite-green solution. Color was developed for 15 mM and absorbance read at 650 nm on a plate reader. Phosphate standards were used to convert absorbance values to picomoles of phosphate released. Phosphatase activity was expressed as percent activity of known substrate (p-Tyr, PTP1B and PTPsigma; PI(3)P, MTMR6).

Immunofluorescence and western blot analyses. U2OS cells were seeded at a density of 35,000 cells per well in McCoy's 5A medium supplemented with 10% FBS on number 1.5 coverglass in 24-well tissue culture plates (for immunofluorescence) or 150,000 cells per well on 6-well dishes (for western blot). After 24 h, siRNAs were transfected at a final concentration of 25 nM using 2 μl HiPerfect transfection reagents (Qiagen) per ml medium. Control siRNA was All-star negative control (Qiagen) and PTPRS siRNAs were two unique sequences (SI02759288, SI03056284, Qiagen). After 48 hr knockdown, cells were treated for 15-60 min by amino acid starvation (cultured in phosphate-buffered saline (PBS) with 10% FBS, 1 g D-glucose per L, MgCl2, and CaCl2), or with rapamycin (50 nM, Calbiochem), chloroquine (25 μM, Sigma), or fresh medium as indicated. For western blots, cells were lysed (in 10 mM KPO4, 1 mM EDTA, 10 mM MgCl2, 5 mM EGTA, 50 mM bisglycerophosphate, 0.5% NP40, 0.1% Brij35, 0.1% sodium deoxycholate, 1 mM NaVO4, 5 mM NaF, 2 mM DTT, AEBSF, aprotinin, bestatin hydrochloride, E64, leupeptin, and pepstatin A and 20 μg of total protein was resolved by SDS-PAGE. Membranes were probed with primary antibodies (LC3B, Cell Signaling Technologies; α-tubulin, Sigma) for 16 h at 4° C. followed by secondary antibodies (HRP-linked anti-rabbit or anti-mouse IgG) for 1 h at room temperature. Proteins were detected by enhanced chemiluminescence. For immunofluorescence, cells were fixed with 3.7% formaldehyde, permeabilized with 0.2% Triton-X 100, and blocked with 3% bovine serum albumin (BSA) in PBS. Antibodies (LC3B, ATG12, and EEA1, Cell Signaling Technologies; V5, Van Andel Institute) were added for 16 h at 4° C. followed by AF-488 conjugated anti-rabbit IgG (Invitrogen) for 1 h at room temperature. Nuclei were counterstained with 2 μg per ml Hoechst-33342 and cells imaged using a 100× oil-immersion objective on a Nikon TE3000 fluorescence microscope (LC3, ATG12) or a 63× water-immersion objective on a Zeiss LSM510 Meta confocal microscope (EEA1, V5).

Transmission electron microscopy (TEM). U2OS cells in 10-cm dishes were transfected with control or PTPRS siRNAs for 48 h. Cells were briefly trypsinized, pelleted, rinsed, and resuspended in 2% glutaraldehyde fixative. Cell pellets were embedded in 2% agarose, postfixed in osmium tetroxide, and dehydrated with an acetone series. Samples were infiltrated and embedded in Poly/Bed 812 resin and polymerized at 60° C. for 24 h. Ultrathin sections (70 nm) were generated with a Power Tome XL (Boeckeler Instruments) and placed on copper grids. Cells were examined using a JEOL 100CX Transmission Electron Microscope at 100 kV. Autophagic structures were quantified from images encompassing approximately 8.5 μm2 of cell area each.

PTPsigma expression. U2OS-2xFYVE-EGFP cells were seeded at a density of 35,000 cells per well in McCoy's 5A medium supplemented with 10% FBS on number 1.5 coverglass in 24-well tissue culture dishes. V5-PTPRS-CTF (BC104812; aa1156-1501) DNA was transfected at 0.2-0.5 μg/well using 2 μl/ml FuGeneHD transfection reagent for 24 h. Cells were fixed with 3.7% formaldehyde, blocked in 3% BSA, and stained with anti-V5 antibodies for 1 h at room temperature. AF546-conjugated anti-mouse-IgG was incubated for 1 h at room temperature and nuclei were stained with Hoechst-33342. Cells were imaged by sequential acquisition using a 63× water immersion objective on a Zeiss LSM510 Meta confocal microscope.

### Example 2

### Identification of PTPsigma as a Phosphatase that Modulates PI(3)P

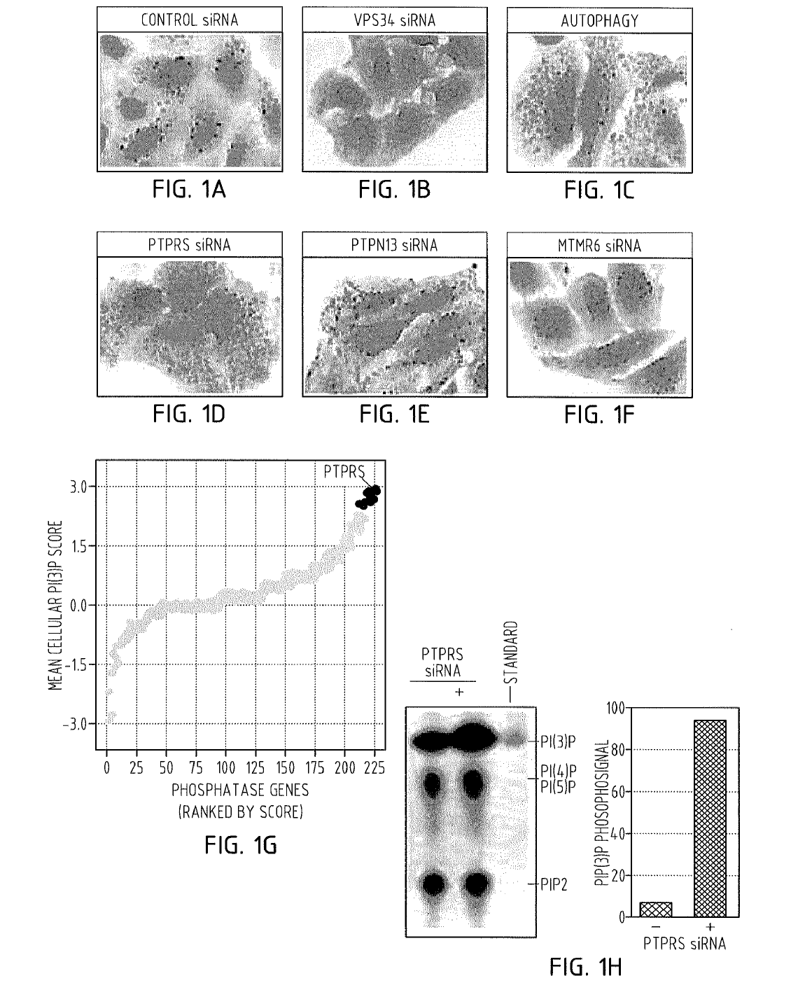

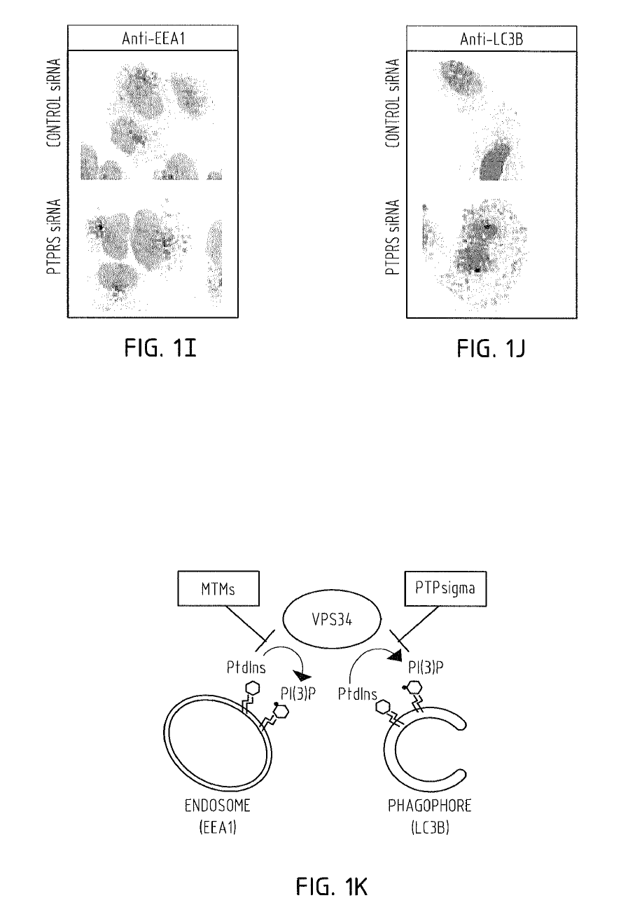

FYVE (Fab1, YOTB, Vac1, and EEA1) domains are cysteine-rich zinc-finger binding motifs that specifically recognize and bind PI(3)P. An EGFP molecule fused to two tandem FYVE domains, termed 2xFYVE-EGFP, serves as an effective cellular sensor for PI(3)P. U2OS cells stably expressing this construct predominantly exhibit punctate PI(3)P-positive endocytic vesicles when cultured in complete growth media and visualized by fluorescence microscopy (FIG. 1A). RNAi-mediated knockdown of Vps34 reduces cellular PI(3)P content and results in a diffuse cytosolic distribution of 2xFYVE-EGFP (FIG. 1B). In contrast, a redistribution of 2xFYVE-EGFP occurs to abundant autophagic vesicles (AVs) when cells are deprived of amino acids to potently induce autophagy (FIG. 1C).

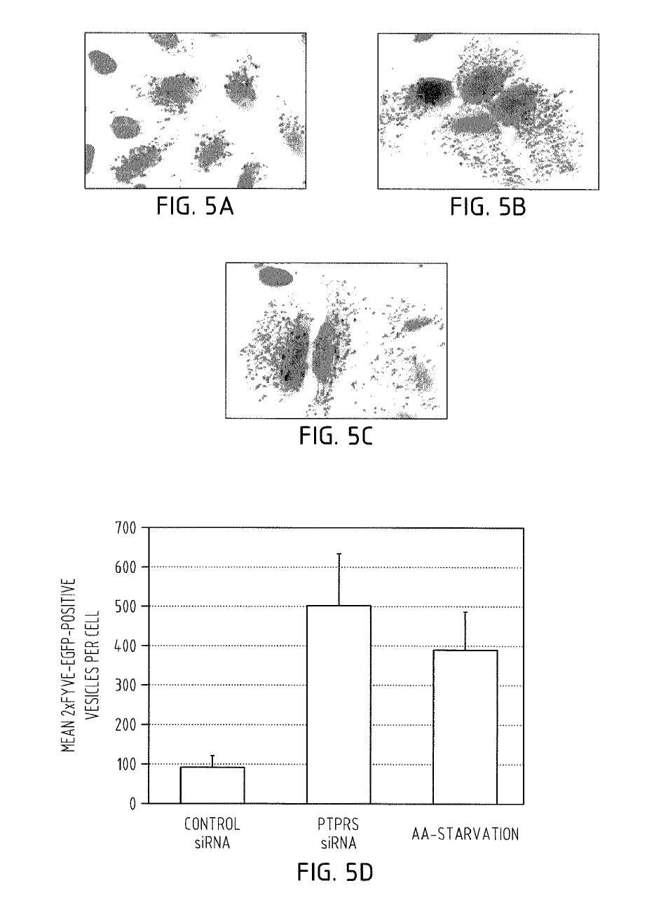

To identify genes that down-regulate PI(3)P signaling, the inventors designed multiple siRNAs targeting over 200 known and putative human phosphatases. The phosphatase siRNAs were introduced into U2OS cells stably expressing 2xFYVE-EGFP and cells were monitored for PI(3)P signaling and autophagy. After minimizing potential off-target effects and validating target knockdown by qRT-PCR, the inventors identified seven genes whose knockdown significantly increased cellular 2xFYVE-EGFP abundance and distribution (FIG. 1G, FIG. 8). In addition to identifying three protein phosphatase regulatory subunits (PPP1R2, PPP2R1B, PPP1R1C), the inventors observed substantial PI(3)P increases following knockdown of the myotubularin family member MTMR6, as well as knockdown of several novel PTPs, including PTPN13 (FAP1) and PTPRS (PTPsigma; or protein tyrosine phosphatase, receptor type, sigma) (FIG. 1D-1F). Knockdown of those phosphatase genes—except for one—was characterized by the appearance of enlarged, frequently perinuclear PI(3)P-positive vesicles. Uniquely, the siRNAs targeting PTPsigma caused a dramatic accumulation of abundant, smaller, autophagic-like double-membrane vesicles throughout the cytosol that phenocopies those seen during autophagy (FIG. 1C, 1D, FIG. 5A-5D). The cDNA sequence for PTPRS (GenBank Accession No. NM_{—}002850) is shown in the concurrently filed Sequence Listing as SEQ ID NO. 1 and the amino acid sequence for PTPsigma is shown in the concurrently filed Sequence Listing as SEQ ID No. 2.

To validate physiological increases in cellular PI(3)P following knockdown of PTPsigma, phospholipids were radiolabeled with ³²PO4 in vivo, extracted, and resolved by thin layer chromatography. Indeed, PI(3)P levels were specifically elevated in the absence of PTPsigma, while other lipid species remained unchanged relative to levels in control cells (FIG. 1H). In order to determine the identity of the PI(3)P-positive vesicles, the inventors immunostained cells with well-established markers of early endosomes (anti-EEA1) and autophagic vesicles (anti-LC3B). The inventors found that knockdown of PTPsigma had no effect on the presence of EEA1-positive endosomes, but significantly increased the abundance of LC3-positive autophagic vesicles (FIG. 1I, 1J). From this, the inventors hypothesized that MTMR6 (and other MTMs, as previously reported) regulate PI(3)P on endosomes, while PTPsigma functions during autophagy (FIG. 1K). On the basis of these results, the inventors focused their attention on PTPsigma as a candidate autophagic lipid phosphatase.

### Example 3

### PTPsigma Negatively Regulates Autophagy

The striking resemblance of PI(3)P-positive vesicles induced by PTPsigma knockdown to autophagic vesicles formed during amino acid starvation led the inventors to propose that autophagy is hyperactivated in the absence of PTPsigma, despite the presence of nutrients. To test this, autophagy was analyzed in U2OS cells by again evaluating LC3 (light chain 3) with antibodies that detect endogenous LC3. LC3 is an ubiquitin-like protein which exists in the cytosol (LC3-I) under normal growth conditions and becomes conjugated to autophagic vesicles (LC3-II) during autophagy. Thus, its aggregation on these autophagic membranes can be analyzed by immunofluorescence. Moreover, the unique electrophoretic mobility of LC3-I and LC3-II allow the isoforms to be separated by SDS-PAGE and assessed using western blot analysis. A caveat of this analysis is that LC3-II is itself degraded in the autolysosome; consequently, LC3-II levels may appear to decrease during very active autophagy when its turnover is most rapid.

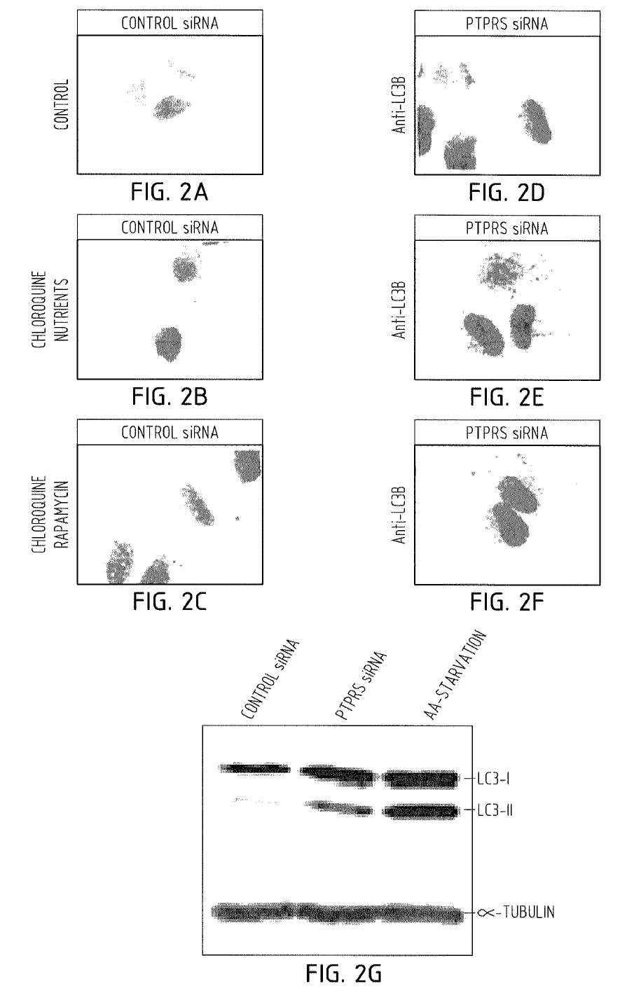

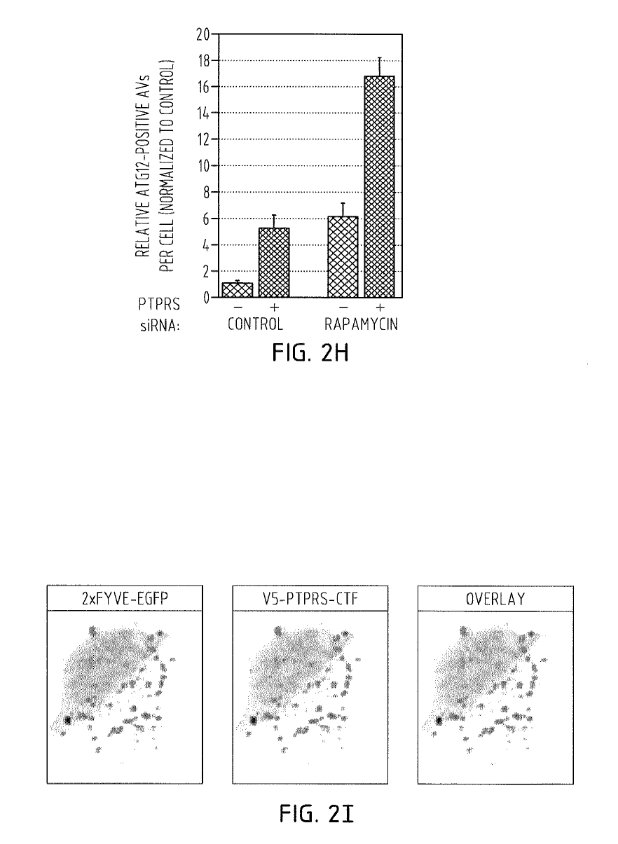

To properly determine LC3 levels, cells were treated with chloroquine, a chemical inhibitor of lysosomal function, which allows LC3-II to form and accumulate to a degree that correlates with the level of autophagic flux. When U2OS cells are cultured in full growth medium (nutrients) and treated with chloroquine for 1 hr, LC3-positive aggregates accumulate (reflecting constitutive AVs) whereas few were seen in control cells (FIG. 2A, 2B). When cells were treated with rapamycin (a potent autophagy inducer) and concurrently supplemented with chloroquine, an even greater abundance of LC3-positive AVs accumulate (FIG. 2C). When these experiments were performed in the absence of PTPsigma, LC3-positive AVs are substantially more abundant under all conditions (FIG. 2D-2F). This same result was captured by western blot analysis of LC3-I and LC3-II isoforms in whole cell lysates (FIG. 2G).

To further confirm hyperactive autophagy in cells lacking PTPsigma, the inventors analyzed ATG12, a second ubiquitin-like molecule that becomes covalently linked to ATG5 on AVs during autophagy. Thus, AVs can be characterized by ATG12-positivity as detected by immunofluorescence. The inventors found that the number of ATG12-positive AVs in PTPsigma knockdown cells was five times that in control cells in the presence of nutrients and three times that in control cells during rapamcyin-induced autophagy (FIG. 2H). Collectively, these results suggest that PTPsigma loss elevates the basal autophagy level and additionally, exacerbates autophagy induced by either starvation or rapamycin treatment.

### Example 4

### PTPsigma Overexpression Reduces Cellular PI(3)P

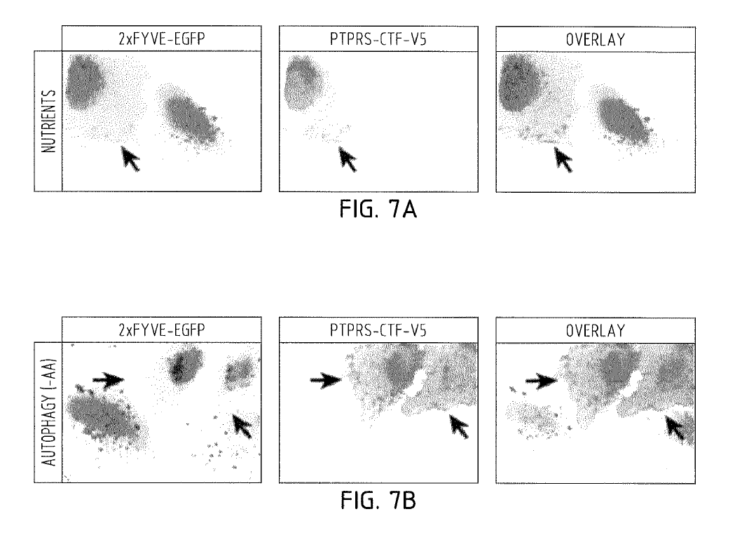

To complement these knockdown studies, the inventors analyzed cellular PI(3)P following exogenous PTPsigma expression. Introduction of the PTPsigma catalytic domains decreased the abundance of PI(3)P-positive vesicles in control cells, notably smaller vesicles throughout the cytosol (FIG. 7A). Importantly, PTPsigma overexpression blunted the production of PI(3)P-positive AVs during amino acid starvation (FIG. 7B). The inventors next assessed the localization of PTPsigma by immunofluorescence in U2OS cells that transiently express V5-tagged PTPRS catalytic domains. This revealed that PTPsigma retains the ability to localize to smaller PI(3)P-positive autophagic vesicles during amino acid starvation (FIG. 2I). These results indicate an active role for PTPsigma in the inhibition of cellular autophagic PI(3)P levels.

### Example 5

### U2OS Cells Lacking PTPsigma and Ptprs−/− MEFs Contain Increased Autophagic Vesicles

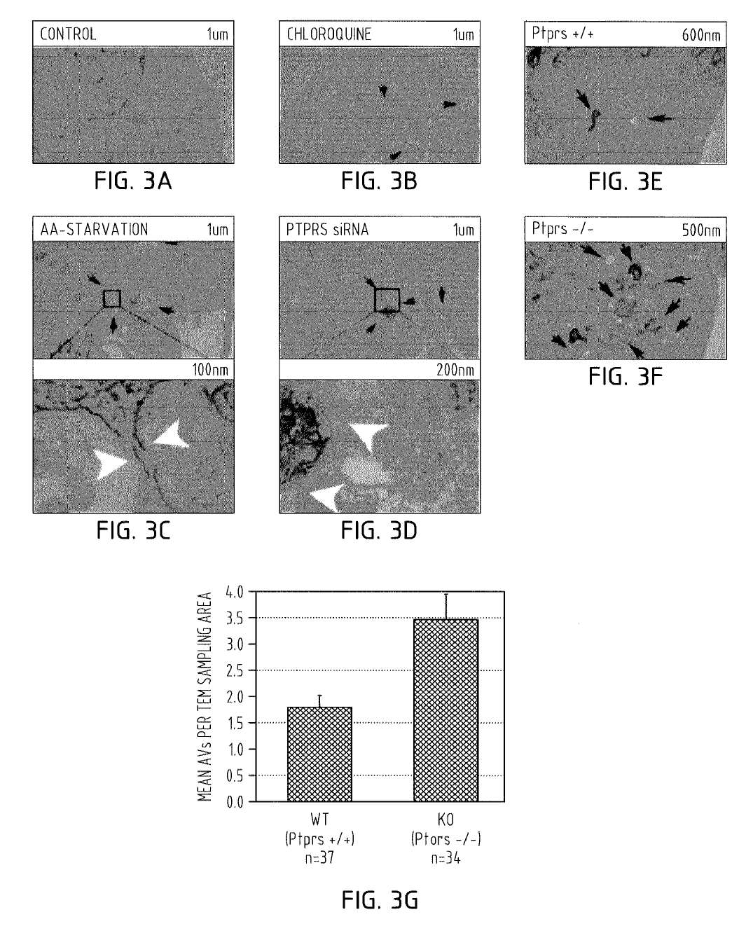

In addition to fluorescent probes, AVs can be detected by transmission electron microscopy (TEM): autophagosomes appear as double-membrane vesicles containing cytosolic components (i.e., organelles and proteins). While few AVs were found in control cells, they were evident in cells treated with chloroquine, as well as in cells deprived of amino acids (FIG. 3A-3C). Similarly, abundant AVs were identified in cells transfected with PTPsigma siRNAs cultured under full growth conditions (FIG. 3D).

To further begin to examine the functional relevance of PTPsigma loss, the inventors analyzed primary wild-type and the knockout (Ptprs−/−) MEFs for their level of autophagy. The inventors have previously generated Ptprs−/− mice by inserting a selectable neomycin resistance gene into the phosphatase domain (aa1399-1518). From these mice the inventors generated primary murine embryonic fibroblasts (MEFs) that lack both Ptprs transcript and protein, as measured by southern blot and western blot, respectively. TEM analysis showed that both wild-type and Ptprs−/− MEFs contained a basal level of autophagic vesicles; however, the autophagosomes were twice as abundant in Ptprs−/− MEFs (FIG. 3E-3G). Collectively, these results suggest that autophagy is physiologically hyperactivated in the absence of PTPsigma.

### Example 6

### PTPsigma Binds and Dephosphorylates PI(3)P in Vitro

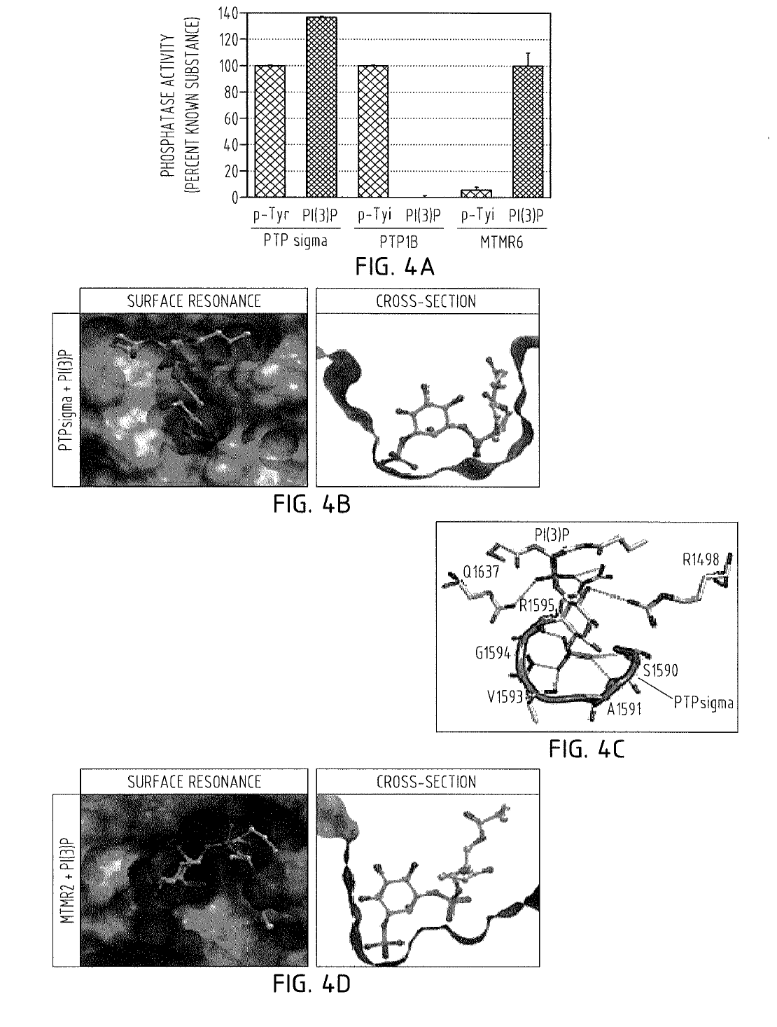

This sizable and specific increase in cellular PI(3)P and the localization of PTPsigma led the inventors to hypothesize that PTPsigma normally serves to dephosphorylate PI(3)P directly. Accordingly, the inventors tested the catalytic activity of PTPsigma (PTPRS-CTF: BC104812 aa1156-1501) against a range of phosphorylated substrates using colorimetric in vitro phosphatase assays. The inventors found that in addition to exhibiting significant activity against a tyrosine-phosphorylated peptide (p-Tyr), PTPsigma also harbored phosphatase activity against PI(3)P (FIG. 4A).

Importantly, PTP1B, a bona fide PTPase, dephosphorylated p-Tyr exclusively and showed no lipid phosphatase activity, while MTMR6 exhibited significant activity against PI(3)P, but only negligible activity against p-Tyr (FIG. 4A). Thus, the ability of PTPsigma to act as a phosphatase against both phosphotyrosine and phosphoinositides is not a universal feature of other PTPs.

A critical feature of lipid phosphatases is a uniquely deep and wide catalytic cleft that accommodates bulky lipid head groups. In particular, the active site of a phosphoinositide phosphatase must be not only large enough to accommodate the hexameric inositol ring, but also wide enough to accommodate the 1′ phosphate that links the ring to a glycerol moiety. To determine if the conformation of either PTPsigma active site would allow PI(3)P binding, the inventors performed structural docking experiments in which a PI(3)P molecule was inserted into the crystal structure of PTPsigma catalytic domains. The inventors discovered that the membrane-proximal D1 domain docked PI(3)P favorably (FIG. 4B), but the membrane-distal D2 domain does not accommodate PI(3)P. The 3′ phosphate is coordinated by the active site residues S1590, A1591, V1593, G1594, and R1595 (FIG. 4C), similar to the binding of a tyrosyl phosphate. The 1′ phosphate of PI(3)P is bound by the side chains of the active site R1595 residue, as well as the R1498 and Q1637 residues that are N-terminal and C-terminal to the active site, respectively. Intriguingly, R1498, which does not contribute to binding of p-Tyr, lies in a less-conserved region near the PTP loop, which is thought to contribute to substrate selectivity. For comparison, the inventors docked PI(3)P into the MTMR2 active site and found that while unique residues contributed to phosphate coordination, the overall size and conformation of the active site is similar to that of PTPsigma (FIG. 4D). Importantly, the PTP1B active site could not dock PI(3)P, owing to its deep yet narrow binding cleft, suggesting that the ability to bind PI(3)P is not a common feature of all PTPs. Taken together, these experiments suggest that PI(3)P is a physiological substrate of PTPsigma.

### Example 7

### Small Molecules Decrease PTPsigma Phosphatase Activity in Vitro

Small molecule inhibitors (10 μM) or sodium orthovanadate (10 mM) were incubated with recombinant PTPsigma for 150 minutes at room temperature in phosphotyrosine assay buffer (25 mM HEPES, 50 mM NaCl, 2.5 mM EDTA, 50 ug/ml BSA, and 10 mM DTT). Para-nitrophenylphosphate (pNPP) was added to a final concentration of 5 mM and reactions performed at 37° C. for 15 m. Para-nitrophenol substrate produced by dephosphorylation was measured spectrophotometrically at 405 nm to determine phosphatase activity. Relative activity was determined by normalizing absorbances to reactions of PTPsigma preincubated with DMSO only. As shown in FIG. 11 (and FIG. 9B), nineteen compounds had some activity, as follows: >50% inhibition in PTPRS activity (RS-49, RS-6, RS-48,RS46, RS-28); 40-50% inhibition in PTPRS activity (RS-32, RS-45, RS-17, RS-36, RS-21, RS-19); 25-40% inhibition in PTPRS activity (RS-15, RS-43, RS-13, RS-11, RS-12); 10-25% inhibition in PTPRS activity (RS-1, RS-34, RS-18). Negative Control=DMSO; and Positive Control=Na3VO4 (known mM inhibitor of PTPs).The chemical structures of these small molecules are shown in FIGS. 9A and 10. The chemical structures of additional small molecule inhibitors derived from small molecule inhibitors RS-6, RS-49, RS-48, and RS-46, are shown in FIGS. 12-15, respectively.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention.

The invention claimed is:

1. A method of treating an autophagy-related disorder in a subject, comprising administering to the subject an effective amount of an agent which modulates expression of the gene encoding protein tyrosine phosphatase receptor type sigma (PTPRS) or the PTPRS gene product (PTPsigma), or which modulates the biological activity of PTPsigma.

2. The method of claim 1, wherein the agent is an antagonist of PTPRS or PTPsigma.

3. The method of claim 1, wherein the autophagy-related disorder is selected from the group consisting of a neurodegenerative disorder, an auto-immune disorder, a cardiovascular disorder, a metabolic disorder, hamartoma syndrome, a genetic muscle disorder, a myopathy, and a cancer.

4. The method of claim 1, wherein the agent is an agonist of PTPRS or PTPsigma.

5. The method of claim 1, wherein the agent is selected from the group consisting of an inhibitory nucleic acid, a small organic molecule, an anti-PTPsigma antibody or antigen-binding fragment thereof, and derivatives thereof.

6. The method of claim 5, wherein the agent is an inhibitory nucleic acid.

7. The method of claim 6, wherein the inhibitory nucleic acid is selected from the group consisting of an siRNA targeting any one of the nucleic acids of SEQ ID NOs: 3-7.

8. The method of claim 5, wherein the agent is a small organic molecule.

9. The method of claim 8, wherein the small organic molecule is a sulfonamide of the formula:
R₁—NH—SO₂—R₂—O—(CH₂)ₙ—CO—NR₃R₄ (I)
where n is 1 thru 3;
where R₁ is:
C₁-C₄ alkyl;
C₃-C₇ cycloalkyl;
phenyl-(CH₂)ₘ— where m is 0 thru 2 and phenyl is optionally substituted with one or two CH₃—, C₂H₅—, F— and Cl—;
phenyl-CH(CH₃)— where phenyl is optionally substituted with CH₃—, C₂H₅—, F— and Cl—;
where R₂ is phenyl optionally substituted with one F—, Cl—, CH₃—, C₂H₅—, and (CH₃)₂CH—;
where R₃ is H—:
where R₄ is:
C₁-C₃ alkyl;
C₃-C₇ cycloalkyl;
—CH₂—CH═CH₂
—(CH₂)_{z}—O—R₅ where z is 1 thru 5 and R₅ is C₁-C₃ alkyl;
—(CH₂)_{w}—R₆ where w is 1 thru 3 and R₆ is tetrahydrofuran or C₃-C₇ cycloalkyl optionally containing one double bond;
—(CH₂)_{w}—R₇ where R₇ is C₁-C₃ alkyl and C₁-C₂ alkoxy and where w is as defined above;
where R₃ and R₄ are taken together with the attached nitrogen atom to form a piperidinyl, piperazinyl, morpholinyl, pyrrolidinyl and pyridinyl ring;
and pharmaceutically acceptable salts thereof.

10. The method of claim 8, wherein the small organic molecule is a pyrazole of the formula: 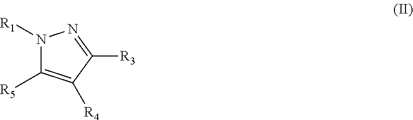
where R₁ is H—, CH₃—, C₂H₅— and cyclo C₃H₅—;
where R₃ is H—, F—, Cl—, Br—, I—, —NO₂, R₃₋₁-phenyl-CO—NH— where R₃₋₁ is CH₃—CO—, CH₃—, C₂H₅—, F—, Cl— and —NO₂;
where R₄ is H—, F—, Cl—, Br—, —NO₂, —CO—O⁻, R₄₋₁-phenyl-CO—NH— where R₄₋₁ is CH₃—CO—, CH₃—, C₂H₅—, F—, Cl— and —NO₂;
where R₅ is H—, F—, Cl—, Br—, I—, —NO₂, R₅₋₁-phenyl-CO—NH— where R₃₋₁ is CH₃—CO—, CH₃—, C₂H₅—, F—, Cl— and —NO₂;
with the proviso:
(1) that one of R₃, R₄ and R₅ must be R₃₋₁-phenyl-CO—NH—, R₄₋₁-phenyl-CO—NH— or R₅₋₁-phenyl-CO—NH—;
and pharmaceutically acceptable salts thereof.

11. The method of claim 8, wherein the small organic molecule is a ketoester of the formula
X₁—CO—O—CHR₁—CO—R₂ (III)
where X₁ is fluoren-9-one;
where R₁ is:
H—,
C₁-C₃ alkyl,
phenyl optionally substituted with one or two
F—,
Cl,
—NO₂;
where R₂ is:
1-naphthyl,
2-naphthyl,
phenyl optionally substituted with one or two
C₁-C₃ alkyl,
C₁-C₂ alkoxy,
F—,
Cl—,
Br—,
—NO₂,
—O—CO-phenyl optionally substituted with 1 F—, Cl— and CH₃—;
and pharmaceutically acceptable salts thereof.

12. The method of claim 8, wherein the small organic molecule is a substituted phenyl compound of the formula 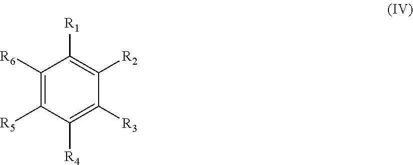
where R₁ is
—CO—CH₃
—CO—NH—R₁₋₁ where R₁₋₁ is
naphthyl
phenyl optionally substituted with one
CH₃—CO—
CH₃—CO—NH—
phenyl-CO—CH═CH—
Br—
Cl—
⁻O—CO—;
where R₂ is —H, C₁-C₂ alkyl, —(CH₂)ₘ-phenyl where m is 1 or 2;
and where R₂ and R₃ are taken together with the atoms to which they are attached for form a phenyl ring optionally substituted with one —Cl, —Br and —CH₃;
where R₃ is —H, C₁-C₂ alkyl, —NO₂,
—CO—NH-phenyl-CO—CH₃,
—NH—CO—R₃₋₁ where R₃₋₁ is
phenyl optionally substituted with —O—CO—CH₃,
C₁-C₃ alkyl,
2-furanyl,
phthalimide,
coumarin,
—O—CH₂-phenyl optionally substituted with one Cl—, Br— and CH₃—,
—SO₂—NR₃₋₂R₃₋₃ where R₃₋₂ is
—H,
C₁-C₃ alkyl and where R₃₋₃ is
C₁-C₃ alkyl,
phenyl optionally substituted with one C₁-C₂ alkyl,
morpholinyl,
piperidinyl,
piperazinyl,
and where R₃ and R₄ are taken together with the atoms to which they are attached and —O—CH₂—O— to form a methylene dioxo ring;
where R₄ is H—, Cl—, Br— and C₁-C₂ alkyl;
and where R₄ and R₃ are taken together with the atoms to which they are attached and —O—CH₂—O— to form a methylene dioxo ring;
where R₅ is H—, C₁-C₂ alkyl, —NH—CO-phenyl, —NH—CO-phenyl-CO—CH₃ and —NH—CO—(C₁-C₂ alkyl);
where R₆ is H— and Cl—;
and pharmaceutically acceptable salts thereof.

13. The method of claim 1, wherein the biological activity is the phosphatase activity of PTPsigma.

14. The method of claim 1, wherein the agent disrupts the interaction between PTPsigma and phosphatidylinositol 3-phosphate [PI(3)P] or phosphotyrosine (p-Tyr) protein.

15. A method of modulating autophagy in a cell, comprising administering to a cell an agent which modulates expression of PTPRS or PTPsigma, or which modulates the biological activity of PTPsigma; whereby autophagy in the cell is modulated.

16. The method of claim 15, wherein the agent is an antagonist of PTPRS or PTPsigma.

17. The method of claim 15, wherein the agent is an angonist of PTPRS or PTPsigma.

18. The method of claim 15, wherein the agent is selected from the group consisting of an inhibitory nucleic acid, a small organic molecule, an anti-PTP sigma antibody or antigen-binding fragment thereof, and derivatives thereof.

19. The method of claim 18, wherein the agent is an inhibitory nucleic acid.

20. The method of claim 19, wherein the inhibitory nucleic acid is selected from the group consisting of an siRNA targeting any one of the nucleic acids of SEQ ID NOs: 3-7.

21. The method of claim 15, wherein the biological activity is the phosphatase activity of PTPsigma.

22. The method of claim 15, wherein the agent disrupts the interaction between PTPsigma and PI(3)P or p-Tyr protein.

23. A method for identifying an agent capable of modulating autophagy in a cell, comprising:
(a) providing (i) a PTPsigma polypeptide, or a PTPsigma homolog capable of binding to PI(3)P, and (ii) a test compound for screening;
(b) mixing, in any order, the PTPsigma polypeptide, or the homolog, and the test compound; and
(c) measuring the biological activity of the PTPsigma polypeptide, or the homolog, in the presence of the test compound as compared to the biological activity of the PTPsigma polypeptide, or the homolog, in the absence of the test compound;
wherein a change in the biological activity of the PTPsigma polypeptide, or the homolog, in the presence of the test compound as compared to the absence of the test compound is indicative of a test compound that is an agent capable of modulating autophagy in a cell.

24. The method of claim 23, wherein the test compound is selected from the group consisting of an inhibitory nucleic acid, a small organic molecule, an anti-PTP sigma antibody or antigen-binding fragment thereof, and derivatives thereof.

25. The method of claim 24, wherein the biological activity is the phosphatase activity of PTPsigma polypeptide or the homolog.

26. A method for identifying a test compound that modulates autophagy comprising
(a) providing (i) a cell comprising a nucleic acid, or a fragment thereof, that encodes PTPsigma, or a PTPsigma homolog capable of binding to PI(3)P, and (ii) a test compound;
(b) contacting the test compound and the cell; and
(c) measuring the expression of the PTPsigma protein, or the homolog, in the presence of the test compound as compared to the expression of the PTPsigma protein, or homolog, in the absence of the test compound;
wherein a change in expression of the PTPsigma protein, or homolog, in the presence of the test compound is indicative of a test compound that modulates autophagy.

27. The method of claim 26, further comprising an additional step of testing for autophagy.

28. The method of claim 27, wherein the test compound decreases autophagy in the cell.

29. The method of claim 27, wherein the test compound increases autophagy in the cell.

30. A method of determining whether a subject is suffering from or is at risk for an autophagy-related disorder, comprising:
(a) providing a biological sample obtained from a subject; and
(b) determining whether the level of expression of PTPRS nucleic acid or PTPsigma polypeptide in the biological sample differs from the PTPRS or PTPsigma level of expression in a comparable biological sample obtained from a healthy subject.

31. A pharmaceutical composition comprising an effective amount of an agent capable of modulating the expression of PTPRS or PTPsigma, or modulating the biological activity of PTPsigma, and a pharmaceutically acceptable carrier.

32. A pharmaceutical composition according to claim 31 wherein the agent is an inhibitory nucleic acid.

33. A pharmaceutical composition according to claim 32 wherein the agent is selected from the group consisting of an siRNA targeting any one of the nucleic acids of SEQ ID NOs: 3-7.
